SEQUENCE LISTING (4 sequences)

SEQ ID NO: 1 - DNA, 1947 bp, Foamy retrovirus
atggccagcggcagcaacgtggaggaatacgagctggacgtggaggccctggtggtgatcctgcgggaccggaacatcccccggaaccccctgcacggcgaggtgatcggcctgcggctgaccgaggggtggtggggccagatcgagcggttccagatggtgcggctgatcctgcaggacgacgacaacgagcccctgcagcggcccagatacgaggtgatccagagggccgtgaacccccacaccatgttcatgatcagcggccctctggccgagctgcagctggccttccaggacctggacctgcccgagggccccctgagattcggccccctggccaacggccactacgtgcagggcgacccctacagcagcagctaccggcccgtgaccatggccgagaccgcccagatgacccgggacgagctggaagatgtgctgaacacccagagcgagatcgagatccagatgatcaacctgctggaactgtacgaggtggagaccagggccctgcggaggcagctggccgagcggagcagcacaggccagggcggcatcagccctggcgcccctagaagcagaccccccgtgagcagcttcagcggcctgcccagcctgcctagcatccccggcatccaccccagagcccccagcccccccagagccacctccacccccggcaacatcccttggagcctgggcgacgacaaccccccctccagcagcttccctggccccagccagcccagagtgagcttccaccccggcaatcccttcgtggaggaagagggccaccggcccagaagccagagccgggagcggcggagagagatcctgcctgcccccgtgcctagcgcccctcccatgatccagtacatccccgtgccccctccccctcccatcggcaccgtgatccccatccagcacatcagaagcgtgaccggcgagccccccagaaaccccagagagatccccatctggctgggccggaacgccccagccatcgacggcgtgttccccgtgaccacccccgacctgcggtgccggatcatcaacgccatcctgggcggcaacatcggcctgagcctgacccctggcgactgcctgacctgggacagcgccgtggccaccctgttcatccggacccacggcaccttccccatgcaccagctgggcaacgtgatcaagggcatcgtggaccaggaaggcgtcgccaccgcctacaccctgggcatgatgctgtccggccagaactaccagctggtgtccggcatcatccggggctacctgcccggacaggccgtggtgaccgccctgcagcagcggctggatcaggaaatcgacgaccagaccagagccgagaccttcatccagcacctgaacgccgtgtacgagatcctgggcctgaatgccaggggccagagcatccgggccagcgtgaccccccagcccaggcccagccggggcagaggcagaggccagaacacctcccggcccagccagggacccgccaacagcggcagaggccggcagagacccgccagcggccagtccaaccggggcagcagcacccagaaccagaaccaggacaacctgaaccagggcggctacaacctgcggcccaggacctaccagccccagagatatggcggcggacggggcaggcggtggaacgacaacaccaacaaccaggaaagccggcccagcgaccagggcagccagacccccaggcccaaccaggccggctccggcgtgaggggcaaccagagccagacaccccggcctgccgccggaagaggcggcaggggcaatcacaaccggaaccagagaagcagcggagccggcgacagcagagccgtgaacaccgtgacccagagcgccacctccagcaccgatgaaagcagcagcgccgtgaccgccgcctctggcggcgaccagcgggactga

SEQ ID NO: 2 - DNA, 3432 bp, Foamy retrovirus
atgaaccccctgcagctgctgcagcccctgcccgccgagatcaagggcaccaagctgctggcccactgggacagcggcgccaccatcacctgcatccccgagagctttctggaagatgagcagcccatcaagaaaaccctgatcaagaccatccacggcgagaagcagcagaacgtgtactacgtgaccttcaaggtgaaaggccggaaggtggaggccgaggtgatcgccagcccctacgagtacatcctgctgtcccccaccgacgtgccctggctgacccagcagcctctgcagctgaccatcctggtgcccctgcaggaataccaggaaaagatcctgagcaagaccgccctgcccgaggaccagaaacagcagctgaaaaccctgttcgtgaagtacgacaacctgtggcagcactgggagaaccaggtgggccaccggaagatccggccccacaacatcgccaccggcgactacccccccaggccccagaagcagtaccccatcaaccccaaggccaagcccagcatccagatcgtgatcgacgacctgctgaagcagggcgtgctgaccccccagaacagcaccatgaatacccccgtgtaccccgtgcccaagcccgacggccggtggcggatggtgctggactaccgggaggtgaacaagacaatccccctgacagccgcccagaaccagcacagcgccggcatcctggccaccatcgtgcggcagaagtacaagaccaccctggacctggccaacggcttctgggcccaccctatcacccccgagtcctactggctgaccgccttcacatggcagggcaagcagtactgctggacccggctgccccagggcttcctgaacagccctgccctgttcaccgccgacgtggtggatctgctgaaagaaatccccaacgtgcaggtgtacgtggacgacatctacctgagccacgacgaccccaaagaacacgtgcagcagctggaaaaggtgttccagatcctgctgcaggccggctacgtggtgtccctgaagaagagcgagatcggccagaaaaccgtggagttcctgggcttcaacatcaccaaagagggcaggggcctgaccgacaccttcaagacaaagctgctgaacatcaccccccccaaggacctgaagcagctgcagagcatcctgggcctgctgaacttcgcccggaacttcatccccaacttcgccgagctggtgcagcctctgtacaacctgatcgcctccgccaagggcaagtacatcgagtggagcgaggaaaacaccaagcagctgaacatggtgatcgaggccctgaacaccgccagcaacctggaagagcggctgcccgagcagcggctggtgatcaaggtgaacaccagccccagcgccggctatgtgcggtactacaacgagaccggcaagaaacccatcatgtacctgaactacgtgttcagcaaggccgagctgaagttctccatgctggaaaaactgctgaccaccatgcacaaggccctgatcaaagccatggatctggccatgggccaggaaatcctggtctacagccccatcgtgagcatgaccaagatccagaaaacccccctgcctgagcggaaggccctgcccatccggtggatcacctggatgacctacctggaagatccccggatccagttccactacgacaagaccctgccagagctgaagcacatccccgacgtgtacaccagcagccagagccccgtgaagcaccccagccagtacgagggcgtgttctacaccgacggcagcgccatcaagtcccccgaccccaccaagagcaacaacgccggcatgggcatcgtgcacgccacctacaagcccgagtaccaggtgctgaaccagtggagcatccccctgggcaaccacaccgcccagatggccgagatcgccgccgtggagtttgcctgcaagaaggccctgaagatccctggccccgtgctggtgattaccgacagcttctacgtggccgagagcgccaacaaagagctgccctactggaagagcaacggcttcgtgaacaacaagaagaagcccctgaaacacatcagcaagtggaagagcatcgccgagtgcctgagcatgaagcccgacatcaccatccagcacgagaaggggcaccagcccaccaacaccagcatccacaccgaaggcaacgccctggccgacaagctggccacccagggctcctacgtggtgaactgcaacaccaagaagcccaacctggacgccgaactggaccagctgctgcagggccactacatcaagggctaccccaagcagtacacctattttctggaagatggcaaggtgaaagtgtccaggcccgagggcgtgaagatcatcccccctcagagcgaccggcagaaaatcgtgctgcaggcccacaacctggcccacaccggcagagaggccaccctgctgaagatcgccaacctgtactggtggcccaacatgcggaaggacgtggtgaaacagctgggccggtgccagcagtgcctgatcaccaacgccagcaacaaggccagcggccccatcctgcggcccgacagaccccagaagcccttcgacaagttcttcatcgactacatcggccccctgccccccagccagggctacctgtacgtgctggtggtggtggacggcatgaccggcttcacctggctgtaccctaccaaggcccccagcacctccgccaccgtgaagagcctgaacgtgctgaccagcatcgccatccccaaggtgatccacagcgaccagggagccgccttcaccagcagcaccttcgccgagtgggccaaagagcggggcatccacctggaattttccaccccctaccacccccagagcagcggcaaggtggagcggaagaacagcgacatcaagcggctgctgaccaaactgctggtgggcaggcccaccaagtggtacgatctgctgcccgtggtgcagctggccctgaataacacctactcccccgtgctgaagtacacccctcaccagctgctgttcggcatcgacagcaacacccccttcgccaatcaggacaccctggatctgacccgggaggaagagctgtctctgctgcaggaaatccggaccagcctgtaccaccccagcaccccccctgccagcagcaggtcctggtcccccgtggtgggccagctggtgcaggaacgggtcgccaggcccgccagcctgagaccccggtggcacaagccctccacagtgctgaaggtgctgaatccccggaccgtggtgatcctggaccacctgggcaacaaccggaccgtgagcatcgacaacctgaagcccacctcccaccagaacggcaccaccaacgacaccgccaccatggaccacctggaaaagaacgagtga

SEQ ID NO: 3 - DNA, 2967 bp, Foamy retrovirus
atggcccctcccatgaccctgcagcagtggatcatctggaagaagatgaacaaggcccacgaggccctgcagaacaccaccaccgtgaccgagcagcagaaagagcagatcatcctggacatccagaacgaggaagtgcagcccaccaggcgggacaagttcagatacctgctgtacacctgctgcgccacctccagccgggtgctggcctggatgttcctggtgtgcatcctgctgatcatcgtgctggtgtcctgcttcgtgaccatcagccggatccagtggaacaaggacatccaggtgctgggccccgtgatcgactggaacgtgacccagcgggccgtgtaccagcccctgcagacccggcggatcgcccggtccctgcggatgcagcaccccgtgcccaagtacgtggaggtgaacatgaccagcatcccccagggcgtgtactacgagccccaccccgagcccatcgtggtgaaagaaagagtgctgggcctgagccagatcctgatgatcaacagcgagaacatcgccaacaacgccaacctgacccaggaagtgaagaaactgctgaccgagatggtgaacgaagagatgcagagcctgagcgacgtgatgatcgacttcgagatccccctgggcgaccccagggaccaggaacagtacatccaccggaagtgctaccaggaatttgccaactgctacctggtgaagtacaaagagcccaagccctggcccaaagagggcctgatcgccgaccagtgccccctgcccggctatcacgccggcctgacctacaaccggcagagcatctgggactactacatcaaggtggagagcatcaggcccgccaactggaccaccaagagcaagtacggccaggcccggctgggcagcttctacatccccagcagcctgcggcagatcaacgtgagccacgtgctgttctgcagcgaccagctgtacagcaagtggtacaacatcgagaacaccatcgagcagaacgagcggttcctgctgaacaagctgaataacctgaccagcggcaccagcgtgctgaagaagagagccctgcccaaggactggtccagccagggcaagaacgccctgttccgggagatcaatgtgctggacatctgcagcaagcccgagagcgtgatcctgctgaataccagctactacagcttcagcctgtgggagggcgactgcaacttcaccaaggacatgatcagccagctggtgcccgagtgcgacggcttctacaacaactccaagtggatgcacatgcacccctacgcctgccggttctggcggagcaagaacgagaaagaggaaaccaagtgccgggacggcgagaccaagcggtgcctgtactaccccctgtgggacagccctgagagcacctacgacttcggctacctggcctaccagaagaacttccccagccccatctgcatcgaacagcagaagatccgggaccaggactacgaggtgtacagcctgtaccaggaatgcaagatcgccagcaaggcctacggcatcgacaccgtgctgttcagcctgaagaatttcctgaactacaccggcacccccgtgaacgagatgcccaacgccagggccttcgtgggcctgattgaccccaagttcccccccagctaccccaacgtgacccgggagcactacaccagctgcaacaaccggaagcggcggagcgtggacaacaactacgccaagctgcggagcatgggctacgctctgacaggcgccgtgcagaccctgtcccagatcagcgacatcaacgacgagaacctgcagcagggcatctacctgctgcgggaccacgtgatcaccctgatggaagccaccctgcacgacatcagcgtgatggaaggcatgttcgccgtgcagcacctgcacacccacctgaatcacctgaaaaccatgctgctggaacggcgcatcgactggacctacatgagcagcacctggctgcagcagcagctgcagaaaagcgacgacgagatgaaggtgatcaagcggatcgccagatctctggtgtactacgtgaagcagacccacagcagccccaccgccaccgcctgggagatcggcctgtactatgagctggtgatccccaagcacatctacctgaacaactggaatgtggtgaacatcggccacctggtgaaaagcgccggacagctgacccacgtgaccatcgcccacccctacgagatcatcaacaaagaatgcgtggagaccatctatctgcacctggaagattgcacccggcaggactacgtgatctgcgacgtggtgaagatcgtgcagccctgcggcaacagcagcgacaccagcgactgccccgtgtgggccgaggccgtgaaagaacccttcgtgcaggtgaaccccctgaagaacggctcctacctggtgctggccagcagcaccgactgccagatccccccctacgtgcccagcatcgtgaccgtgaatgagaccacctcctgcttcggcctggacttcaagcggcccctggtggccgaggaaagactgagcttcgagccccggctgcccaacctgcagctgaggctgccccacctggtgggcatcatcgccaagatcaagggcatcaagatcgaggtgaccagcagcggcgagagcatcaaagaacagatcgagcgggccaaggccgagctgctgcggctggatatccacgagggcgacacacccgcctggatccagcagctggccgccgccaccaaggacgtgtggcccgctgcagccagcgccctgcagggcatcggcaactttctgagcggcaccgcccagggcatcttcggcaccgccttctccctgctgggctacctgaagcccatcctgatcggcgtgggcgtgattctgctggtgattctgatcttcaagatcgtgagctggatccccaccaagaaaaagaaccagtga

SEQ ID NO: 4 - DNA, 2970 bp, Foamy retrovirus
atggcccctcccatgaccctggaacagtggctgctgtggaagaagatgagccaggcccaccaggccctggaaaacgtgaccaccctgaccgaggaacagaaacagcaggtgatcatcgacatccagcacgaggacgtggtgcccacccggatggacaagctgaagtacctggcctacagctgctgcgccacctccacccgggtgctgtgctggatcgtgctggtgtgcgtgctgctgctggtggtgttcatcagctgcttcgtgaccatgagccggatccagtggaacaaggacatcgccgtgttcggccccgtgatcgactggaacgtgagccagcaggccgtgatccagcagatccgggccaagcggctggccagatccatccgggtcgagcacgccaccgagacctacgtggaggtgaacatgaccagcatcccccagggcgtgctgtacgtgccccaccccgagcccatcatcctgaaagaacgggtgctgggcctgagccaggtgatcatgatcaacagcgagaacatcgccaacaccgccaacctgacccaggaaaccaaggtcctgctggccgacatgatcaacgaggaaatgaacgacctggccaaccagatgatcgacttcgagatccccctgggcgaccccagggaccagaagcagtaccagcaccagaagtgcttccaggaatttgcccactgctacctggtgaagtacaagaccaccaagggctggcctagcagcaccgtgatcgccgaccagtgccccctgcccggcaaccaccccaccgtgcagtacgcccaccagaacatctgggactactacgtgcccttcgagcagatcaggcccgagggctggaacagcaagagctactacgaggacgcccggatcggcggcttctacatccccaagtggctgcggaacaacagctacacccacgtgctgttctgcagcgaccagatctacggcaagtggtacaacatcgacctgaccgcccaggaacgggagaacctgctggtgcagaagctgatcaacctggccaagggcaacagcagccagctgaaggacagagccatgcccgccgagtgggacaagcagggcaaggccgacctgttccggcagatcaacaccctggacgtgtgcaaccggcccgagatggtgttcctgctgaacagctcctactacgagttcagcctgtgggagggcgactgcggcttcacccggcagaacgtgacccaggccaacagcctgtgcaaggacttctacaacaacagcaagtggcagaagctgcacccttacagctgccggttctggcggtacaagcaggaaaaagaagagaccaagtgcagcaacggcgagaagaagaagtgcctgtactacccccagtgggacacccctgaggccctgtacgacttcggcttcctggcctacctgaacagcttccccagccccatctgcatcaagaaccagaccatccgggagcccgagtacgagatcagcagcctgtacctggaatgcatgaacgccagcgaccggcacggcatcgacagcgccctgctggccctgaaaaccttcctgaacttcaccggccagagcgtgaacgagatgcccctggccagggccttcgtgggcctgaccgaccccaagttcccccccacctaccccaacatcacccgggagagcagcggctgcaacaacaacaagcggaagcggcggagcgtgaacaactacgagcggctgcggagcatgggctacgctctgacaggcgccgtgcagaccctgagccagatcagcgacatcaacgacgagaggctgcagcacggcgtgtacctgctgcgggaccacgtggtgaccctgatggaagccgccctgcacgacgtgagcatcatggaaggcatgctggccattcagcacgtgcacacccacctgaatcacctgaaaaccatgctgctgatgaggaagatcgattggaccttcatcagaagcgactggatccagcagcagctgcagaaaaccgacgacgagatgaagctgatccggcggaccgcccggtccctggtgtactacgtgacacagaccagcagctcccccaccgccacaagctgggagatcggcatctactacgagatcgtgatccccaagcacatctacctgaataactggcaggtgatcaacgtgggccacctgctggaaagcgccggacacctgacccacgtgaaggtgaaacacccctacgagatcatcaacaaagagtgcagcgacacccagtacctgcacctggaagagtgcatccgggaggactacgtgatctgcgacatcgtgcagattgtgcagccctgcggcaatgccaccgagctgtccgactgccccgtgaccgccctgaaagtgaaaaccccctacatccaggtgtcccccctgaagaacggctcctacctggtgctgtccagcaccaaggactgcagcatccccgcctacgtgcccagcgtggtgaccgtgaatgagaccgtgaagtgcttcggcgtggagttccacaagcccctgtacgccgagaccaagaccagctacgagccccaggtgccacacctgaagctgcggctgccccacctgaccggcatcatcgccagcctgcagagcctggaaatcgaggtgaccagcacccaggaaaacatcaaggaccagatcgagcgggccaaggcccagctgctgaggctggacatccacgagggcgacttccccgactggctgaagcaggtggccagcgccaccagggacgtgtggcctgccgccgccagcttcatccagggcgtgggcaacttcctgagcaacaccgcccagggcatcttcggcagcgccgtgagcctgctgtcctacgccaagcccatcctgatcggcatcggcgtgattctgctgatcgccctgctgttcaagatcatcagctggctgcctggcaagcccaagaagaactga

(19) United States
(12) Patent Application Publication
(10) Pub. No.: US 2012/0141440 A1
(43) Pub. Date: Jun. 7, 2012

(54) FOAMYVIRUS VECTORS AND METHODS OF USE

(76) Inventors: Axel Rethwilm, Wurzburg (DE); Dirk Lindemann, Drersden (DE); Helmut Hanenberg, Indianapolis, IN (US); Wade D. Clapp, Indianapolis, IN (US)

(21) Appl. No.: 13/259,190

(22) PCT Filed: Mar. 26, 2010
(86) PCT No.: PCT/US2010/028854

§ 371 Date: Jan. 12, 2012

Related U.S. Application Data
(60) Provisional application No. 61/161,673, filed on Mar. 19, 2009.

(51) Int. Cl. A61K 48/00; C12N 5/10; A61P 7/06; C12N 15/63

(52) U.S. Cl. 424/93.21; 435/320.1; 435/366; 435/325

(57) ABSTRACT

The disclosure of the present application provides foamyvirus vectors, as well as methods and kits involving the same. In at least one embodiment, a plurality of recombinant vectors comprises an expression sequence encoding at least one component of a foamyvirus particle, wherein at least one codon of the expression sequence is optimized for expression in homo sapiens.

### CROSS REFERENCE TO RELATED APPLICATIONS

The present application is an International Patent Application which claims priority to U.S. Provisional Patent Application Ser. No. 61/163,673, filed Mar. 26, 2009, which is incorporated herein by reference

### STATEMENT REGARDING FEDERALLY SPONSORED RESEARCH AND DEVELOPMENT

Part of the work during the development of this present disclosure was made with government support from the National Institutes of Health under grant number PPG-P01-HL533586. The U.S. Government has certain rights in the present disclosure.

### BACKGROUND

Treatment of severe combined immunodeficiencies and chronic granulomatosis disease with genetically corrected hematopoietic stem cells (HSC) are the two genetic therapies to date that have been proven to be highly effective in humans. As efficient transformation of the stem cells are required for these theapies, retroviral vectors, such as gammaretroviral (GR) vectors or lentiviral-based vectors have been examined. Unfortunately, both of these vector systems are based on wild-type viruses that are associated with severe pathogenicity in their natural hosts.

Fanconi Anemia (FA) is a complex recessive inherited disorder that is clinically characterized by variable congenital abnormalities, progressive bone marrow (BM) failure and a high propensity to develop myeloid and epithelial malignancies. On a cellular level, FA is characterized by a profound hypersensitivity upon exposure to DNA cross-linking agents such as mitomycin-C (MMC) or diepoxybutane (DEB). Genetically, germ-line mutations in thirteen genes (FANCA/B/C/D1/D2/E/F/G//I/J/L/M/N) result in the clinical phenotype of FA. The only long-term cure for the BM failure in FA patients is transplantation of normal hematopoietic stem cells, ideally from an HLA-matched sibling. Allogeneic BM or cord blood transplantation is not without subsequent risk as the conditioning regimens cause genotoxic stress that predispose patients to an increased incidence of squamous cell carcinomas especially when compounded by the presence of chronic graft-versus-host disease. Thus, the ability to transduce autologous, genetically corrected stem cells in the absence of genotoxic myelopreparation, which could provide a therapy that does not expose the patient to these ongoing potential sequelae, is desired. Unfortunately, the gene therapy trials of FANCC−/− and FANCA−/− patients have not shown evidence of long-term presence of corrected cells in vivo and have not shown any clinical benefits for the patients

Given the increasing identification of diseases, such as FA, which are tied to a genetic defect, there is a need for genetic therapy vectors and methods of using the vectors for the correction of genetic defects. Additionally, there is a need that the vectors used for gene therapy techniques be free, or minimized, of the potential for causing secondary sequelae.

### SUMMARY

Disclosed herein are compositions, cells, kits and methods involving foamyvirus vectors. In at least one embodiment of the present diclosure, a foamyvirus expression system comprises a plurality of recombinant vectors, where the plurality of recombinant vectors have an expression sequence encoding at least one component of a foamyvirus particle, wherein at least one codon of the expression sequence is optimized for expression in homo sapiens. The expression sequence may be selected from a group consisting of SEQ. ID. NOS. 1-4, an optimized sequence at least about 75% identical to one of SEQ. ID. NOS. 1-4, an optimized sequence at least about 85% identical to one of SEQ. ID. NOS. 1-4, an optimized sequence at least about 95% identical to one of SEQ. ID. NOS. 1-4, and an optimized sequence at least about 99% identical to one of SEQ. ID. NOS. 1-4. Further, the expression sequence may be a fragment of one of SEQ. ID. NOS. 1-4, wherein the fragment has a length selected from a group consisting of at least 250 nucleotides, at least 500 nucleotides, at least 750 nucleotides, at least 1000 nucleotides, at least 1250 nucleotides, at least 1500 nucleotides, at least 1750 nucleotides, and at least 2000 nucleotides.

In an embodiment of the present disclosure, the plurality of recombinant vectors comprise a first vector and a second vector, the first vector having an RNA packaging signal, and the second vector having an env expression sequence. The plurality of recombinant vectors may also comprise a third vector having a gag expression sequence, and optionally a pol expression sequence. Additionally, the plurality of recombinant vectors may comprise a fourth vector having a pol expression sequence. Further, in at least one embodiment, the plurality of recombinant vectors may further comprise a non-foamyvirus expression sequence, such as Fancg,

In at least one embodiment of the present disclosure, the foamyvirus expression system having a plurality of recombinant vectors may be capable of producing a foamyvirus particle following transfection into an acceptable host. The expression system, in at least one embodiment, is capable of producing at least about 10, or alternately 100, times as much foamyvirus particles as compared to an non-optimized foamyvirus expression system.

In at least one embodiment of the expression system, the at least one component of a foamyvirus particle comprises a gag protein, a pol protein, and an env protein.

In at least one embodiment of the expression system, the system produces at least about 2 times, or at least about 4 times, as much gag protein as compared to a system with a non-optimized expression sequence.

In at least one embodiment of the expression system, the system produces at least about 2 times, at least about 4 times, at least about 16 times, or at least about 64 times as much pol protein as compared to a system with a non-optimized expression sequence.

In at least one embodiment of the expression system, the system produces at least about 4 times, at least about 16 times, at least about 64 times, at least about 128 times, at least about 256 times, or at least about 512 times as much env protein as compared to a system with a non-optimized expression sequence.

In an exemplary embodiment of the foamyvirus expression system, the system comprises a plurality of recombinant vectors, wherein the plurality of recombinant vectors comprise at least one nucleotide sequence, wherein the at least one nucleotide sequence has at least about 75% nucleotide identity to SEQ. ID. NOS. 1-4, and wherein the plurality of recombinant vectors are capable of producing a foamyvirus particle when transformed into an appropriate host cell.

In at least one embodiment of the disclosure, a transformed cell comprises an embodiment of the foamyvirus expression system described herein. Additionally, the transformed cell may be capable of compensating for a genetic defect. Further, the transformed cell may be obtained from a patient having the genetic defect prior to transformation with the expression system.

In at least one embodiment of the disclosure, a kit for the expression of foamyvirus in mammalian cells comprises a plurality of recombinant vectors having a foamyvirus expression sequence, which is optimized for expression in homo sapiens. The plurality of recombinant vectors may further comprise a first vector, a second vector, a third vector, and a fourth vector. In an exemplary embodiment of the kit, the first vector comprises a viral packaging signal, the second vector comprises a sequence encoding gag, the third vector comprises a sequence encoding pol, and the fourth vector comprises a sequence encoding env. Further, in an embodiment of the kit, the concentration of the first vector is greater than the second vector, the concentration of the second vector is greater than the third vector, the concentration of the third vector is greater than the fourth, and the plurality of recombinant vectors is capable of producing foamyvirus particles when transfected into mammalian cells. In at least one embodiment, the ratio of first vector to second vector to third vector to fourth vector may be about 10 to about 5 to about 1 to about 1 respectively.

In at least one embodiment of a method of use of a foamyvirus expression system, the method comprises introducing a plurality of recombinant vectors having an optimized foamyvirus expression sequence into a host cell, introducing the host cell containing the plurality of recombinant vectors into a host organism, wherein the plurality of recombinant vectors is operable to produce foamyvirus particles after introduction into the host cell. Further, the plurality of recombinant vectors in the method may be capable of compensating for a genetic defect, such as one resulting in Fanconi Anemia, in a host organism.

### BRIEF DESCRIPTION OF THE DRAWINGS

FIG. 1 shows a depiction of the genetic organization of the prototype foamyvirus (PRF);

FIG. 2 shows a depiction of the genetic organization of foamyvirus vectors for the 2-/3-/4-plasmid systems, according to at least one embodiment of the present disclosure;

FIG. 3 shows a graphical depiction of titer levels for the 2-, 3-, and 4-plasmid systems, according to at least one embodiment of the present disclosure;

FIG. 4 shows a depiction of packaging/helper constructs for the 2, 3, and 4 plasmid system, according to at least one embodiment of the present disclosure;

FIG. 5 shows a depiction of at least one strategy for destruction of functional groups in three Foamyvirus genes, according to at least one embodiment of the present disclosure;

FIG. 6 shows a depiction of the genetic structure of the final packaging constructs, according to at least one embodiment of the present disclosure;

FIG. 7 depicts a western blot analysis of protein expressed from wild-type and human codon optimized foamyvirus gag, pol, and env genes in expression constructs in 293T cells, according to at least one embodiment of the disclosure;

FIG. 8 shows the relative ratio of foamyvirus gag and pol for the production of foamyvirus particles, according to an embodiment of the disclosure;

FIG. 9 shows the optimization of the amount of vector (puc2MD9) and env plasmids (pcoPE), according to an embodiment of the disclosure;

FIG. 10 shows an optimization of different env mutants for the production of transducing units/ml, according to an embodiment of the disclosure;

FIG. 11 shows a FACS analysis of wild-type and Fancg−/− CD117+ cells five days post-transfection, according to an embodiment of the disclosure;

FIG. 12A is a graphical illustration of the in vitro gene transfer efficiency in % EGFP+ cells from Fancg−/− mice in liquid expansion, according to an embodiment of the disclosure;

FIG. 12B is a graphical illustration of the in vitro gene transfer efficiency in % EGFP+ cells from Fancg−/− mice in a progenitor assay, according to an embodiment of the disclosure;

FIG. 13 shows a genetic structure of the human FANCG expressing foamyviral MD9 vectors, according to at least one embodiment of the disclosure;

FIG. 14 is a graphical illustration of the relative level of MMC hypersensitivity of transduced Fancg−/− clonogenic CD117+ cells plated in a standard progenitor assay, according to an embodiment of the disclosure;

FIG. 15 is a graphical illustration of the correction of MMC-induced cells arrest in primary human FA-G fibroblast cells by genetic therapy with FV vectors expressing FANCG, according to an embodiment of the disclosure;

FIG. 16 shows the results of a FACS analysis of CD45.2+ cells, according to an embodiment of the disclosure;

FIG. 17 is a graphical illustration of chimerism analysis of mice cotransplanted with CD45.1+ and CD45.2+ cells, according to an embodiment of the disclosure;

FIG. 18 is a graphical illustration of a chimerism analysis of mice two months after receiving secondary transplants, according to an embodiment of the disclosure;

FIG. 19A shows a schematic representation of the foamyviral genome with the viral genes gag, pol, env, Tas, and Bet;

FIG. 19B shows a schematic representation of the recombinant foamyviral vectors MD9-FANCC/EGFP and MD9-EGFP with deletions in gag, pol, env, and the U3 region of the 3′LTR, according to an embodiment of the disclosure;

FIGS. 20A-C show the transduction of wild-type and Fancc−/− CD117+ cells with foamyviral vectors under light (FIG. 20A) and dark field (FIG. 20B) microscopy, and through graphical illustration of the transduction efficiency, according to an embodiment of the disclosure;

FIG. 21 is a western blot analysis of the FANCD2 (A), MMC (B and D), and TNF-a (C and E) for wild-type MD9-EGFP, Fancc−/− MD9-EGFP, and Fancc−/−MD9-FANCC-EGFP, according to an embodiment of the disclosure;

FIGS. 22A-D are depictions of chimerism of mice transplanted with transduced test cells in competitive repopulation assays, as determined by flow cytometry (FIG. 22 A-B) after staining with antibodies against CD45.1 and CD45.2, and by analysis of genotype and transduction efficiency of myeloid progenitors (FIGS. 22 C-D), according to an embodiment of the disclosure; and

FIGS. 23A-B are graphical illustrations (FIG. 23A) and histological visualizations (FIG. 23B) of TNF-α hypersensitivity in primary recipients of mock-transduced Fancc−/− stem cells six months post-transplantation.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the present disclosure is thereby intended.

The present disclosure relates to viral vectors as well as methods of production and uses of the same. Specifically, embodiments of the present disclosure relate to foamyvirus (FV) vectors and methods of use. As used herein, a FV vector may be any foamyvirus vector including, but not limited to, human foamy virus, simian foamy virus, Bovine foamy virus, Equine foamy virus, and Feline foamy virus.

### A. Prototype Foamyvirus

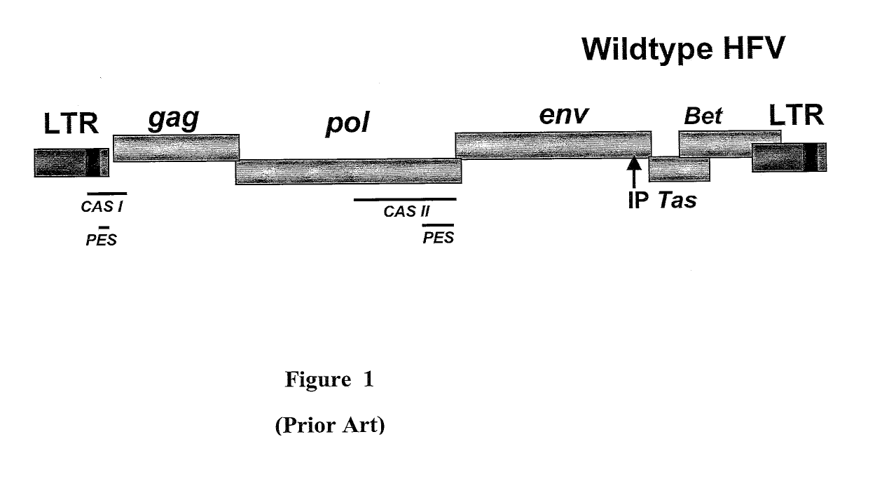

The structure of the prototype foamyvirus (PFV), formerly also called human FV (HFV), is shown in FIG. 1. Although the genetic map looks like one from a typical complex retrovirus with the orderly appearance of gag, pol and env, unique biological features and distinct replication strategies puts FV between orthoretroviruses (all retroviridae except FV) and hepadnavirus such as hepatitis B virus.

In FV, the complete RNA packaging signal is located in two distinct regions that have been named cis-acting region I and II (CAS I and II). CAS I is located in the beginning of the viral genome spanning the R, U5 and the beginning of gag. The CAS II region is located at the 3′ end of pol, close to the start of the env gene. Overlapping with the CAS I and II regions are two functional domains that are instrumental to include the pol gene product into the viral particles, termed pol encapsidation signals (PES). These structural domains of FV are unique among retroviridae as FV are the only members where the pol gene is expressed from a spliced message independent from gag. Pol is included in the virus by specific interactions with the PES sequences of the mRNA genome which binds to gag.

This unique biological feature of FV leads to specific structural requirements for the design of the FV vectors and the packaging plasmids. To be incorporated in the producer cell in the emerging viral particle initially formed by interactions between gag and env, the vector has to contain the two CAS I/II regions thereby interacting with the gag protein as well as the PES domains thereby binding the pol protein.

### B. Human Optimized Genes

Since codon usage varies from organism to organism, a gene utilizing the preferred codon for a specific organism may increase the respective level of protein expressed by the gene's transcript, In at least one embodiment of the present disclosure, the codon usage of at least one of the gag, pol, and env genes are adapted to the codon bias of Homo sapiens. The codon bias of at least one of the gag, poi, and env genes, for regions of GC content between approximately 80% and approximately 30%, may be approximately 100%, about 90% to about 99, or about 75% to about 99% of that of Homo sapiens.

Sequence Listings:
SEQ. ID, No. 1—Homo sapiens optimized Gag cDNA from PFV
SEQ. ID. No. 2—Homo sapiens optimized Pol cDNA from PFV
SEQ. ID. No. 3—Homo sapiens optimized Env cDNA from PFV
SEQ. ID, No. 4—Homo sapiens optimized Env cDNA from Simian Foamy Virus (SFV-1)

In at least one exemplary embodiment, the optimized nucleotide sequence for gag, pol, and env may be a fragment of SEQ. ID. No. 1, SEQ. ID. No. 2, or SEQ. ID. No. 3 respectively. Additionally, env may also be a fragment of SEQ. ID. No. 4. The nucleotide sequence fragment may be at least 250, at least 500, at least 750, at least 1000, at least 1250, at least 1500, at least 1750, at least 2000 nucleotides (nt) or about all of the optimized sequence (one of SEQ. ID. Nos. 1-4). Further, the optimized nucleotide sequence may be a complementary sequence of any of the fragments of SEQ. ID. NO. 1-4 as described above. Moreover, the protein product of at least one embodiment of the optimized nucleotide sequence may have substantially the same activity as the non-optimized sequence.

In an exemplary embodiment, a vector containing a human optimized expression sequence to be tested (gag, pol, or env) with splice donor and acceptor sites (the “Optimized Vector”) was compared to the same parent vector with a non-optimized expression sequence without splice donor and acceptor sites (the “Non-Optimized Vector”). For at least this comparison between Optimized Vector and the Non-Optimized Vector when examined in 293T cells, levels of gag protein may be increased at least approximately 4-fold. Additionally, levels of gag protein may be increased at least 2-fold to at least 4-fold. In the vector comparison, levels of pol protein may be increased as well to a level at least 64-fold. Further, levels of pol protein may be increased at least 16-fold to at least 64-fold. Further still, levels of pol protein may be increased at least 2-fold to at least 64-fold. In the vector comparison, levels of the env protein may be increased by at least 512-fold. Additionally, levels of the env protein may be increased by between approximately 64-fold and 512-fold. Further, levels of the env protein may be increased by approximately 4-fold to 512-fold.

C. 2-/3-/4-Plasmid Systems

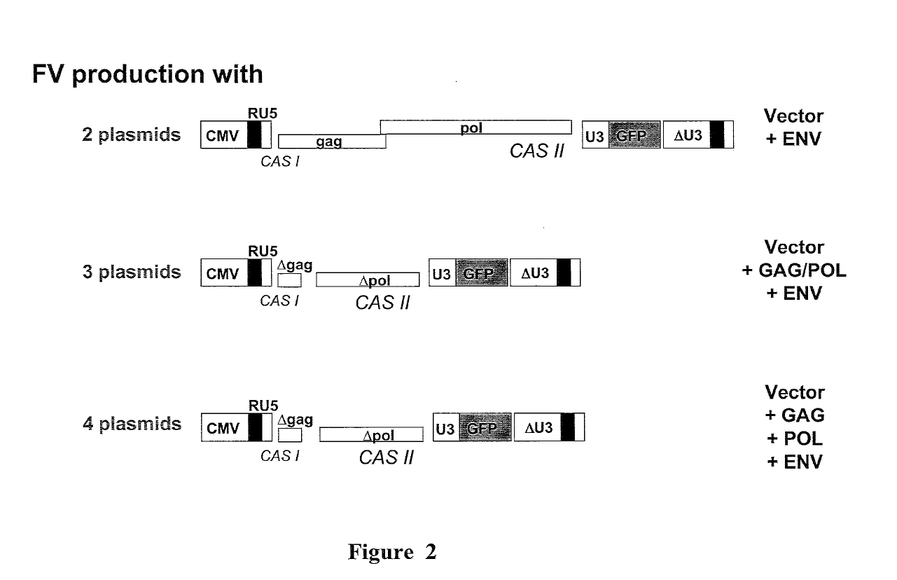

Turning to FIG. 2, various embodiments are depicted for the 2-/3-/4-plasmid systems. In the 2-plasmid system, FV gag and poi are encoded by the transfer plasmid and env is provided in trans from a second plasmid. In the 3- or 4-plasmid systems gag and pol are removed from the transfer plasmid and expressed either from one or two separate packaging plasmids.

In at least one embodiment, the foamyvirus expression system comprises a plurality of recombinant vectors. The plurality of recombinant vectors comprise expression sequences, which encode at least one component of a foamyvirus particle. The expression sequence in this system may be partially or completely optimized for expression in homo sapiens. Further, the plurality of recombinant vectors may comprise a first vector and a second vector, where the first vector has an RNA packaging signal and the second vector has an env expression sequence. The RNA packaging signal has cis-acting regions I and II, and pol encapsidation signals.

The plurality of recombinant vectors, in a further embodiment, may comprise a third vector which has a gag expression sequence, and optionally a pol expression sequence. Additionally, the plurality of recombinant vectors may also include a fourth vector comprising a pol expression sequence. Each of the embodiments of the plurality of recombinant vectors discussed herein may be capable of producing a foamyvirus particle when introduced into an acceptable host.

In at least one embodiment of the foamyvirus expression system, the plurality of recombinant vectors further comprise a non-foamyvirus expression sequence. This non-foamyvirus expression sequence may include any mammalian, or mammalian optimized, sequence that is functional to produce an RNA product, a protein, or a DNA segment. In at least one embodiment, the non-foamyvirus expression sequence may encode Fancg. Further, the non-foamyvirus expression sequence may encode a reporter product which is effective in a mammalian cell. For example, the reporter product may be Green Fluorescent Proein (GFP), any variant of GFP (such as EGFP, BFP, RFP), β-galactosidase, Luciferease, and the secreted variant of alpha-amylase from Bacillus stearothermophilus (SAMY).

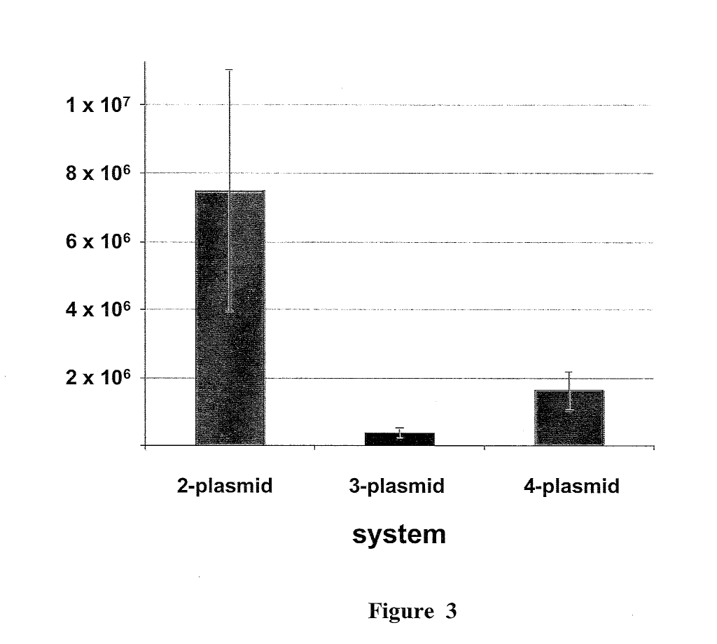

In at one embodiment, the foamyvirus expression system is a highly efficient gene transfer system achieving titers up to 10⁷ infectious particles (FIG. 3) and may be used to genetically modify repopulating CD34+ bone marrow cells from a human, non-human primate (such as common marmosets), domesticated animal, farm animal, or aquatic animal, where marked progeny may be still detectable more than a year after reinfusion.

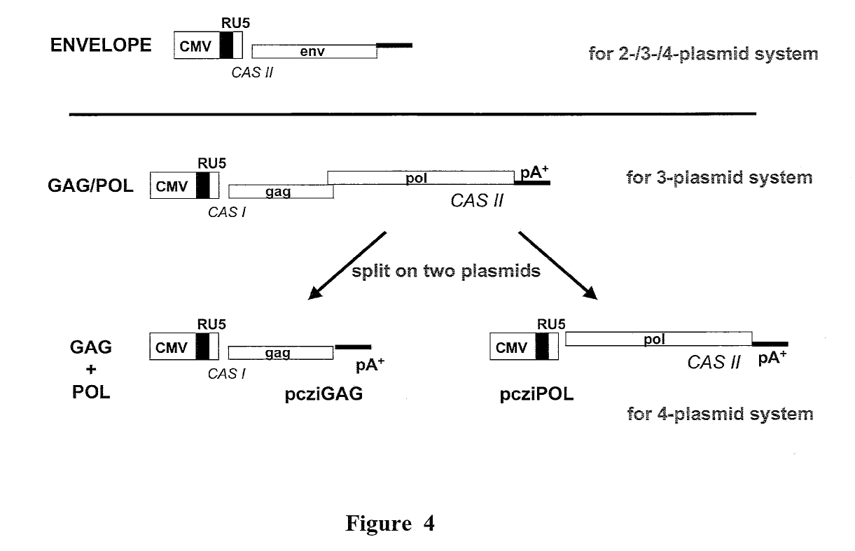

The gag and pol genes may be provided in trans on a second plasmid as shown for the 3- and 4-plasmid system instead of the vector plasmid (FIG. 3). In at least one exemplary embodiment, separating gag and pol onto two different particles in the 3- or 4-plasmid system improves titers and prevents the transfer of genetic material (the packaging plasmid) for expression of pol and gag in the target cells (FIG. 4). The increase in titer for the 4-plasmid compared to the 2- and 3-plasmid is shown in FIG. 3.

The packaging plasmids for the 4-plasmid system however still contain CAS I and PES I in the gag plasmid and CAS II and PES II in case of the pol plasmid. In addition, the internal promotor (IP) for the initiation of the transactivator Tas is also present within the coding region of the env gene. In at least one embodiment of the plurality of recombinant vectors, any overlap between the FV gag, pol and env genes was eliminated, rendering recombination events (almost) impossible, and at the same time improving the protein expression each of the packaging plasmids. Further, in at least one embodiment, one or more of gag, pol and env genes have had one or more of their codons optimized for human expression, as discussed in greater detail above.

### D. Kits for Foamyvirus Expression

In at least one embodiment, a kit for the expression of foamyvirus in mammalian cells includes an embodiment of plurality of recombinant vectors as described herein. In an exemplary embodiment, the plurality of recombinant vectors may include a first vector, a second vector, a third vector, and a fourth vector. The first vector comprises a viral packaging signal, the second vector comprises a sequence encoding gag, the third vector comprises a sequence encoding pol, and the fourth vector comprises a sequence encoding env. In at least one embodiment, the concentration of the first vector is greater than the second vector, which is greater than the concentration of the third vector which is greater than the fourth. Further, in at least one embodiment, the ratio of first vector to second vector to third vector to fourth vector is about 10 to about 5 to about 1 to about 1 respectively. Further, in at least one embodiment, the plurality of recombinant vectors are capable of producing foamyvirus particles when transfected into mammalian cells.

### E. Methods

In at least one embodiment, a method of use of a foamyvirus expression system comprises introducing a plurality of recombinant vectors into a host cell, introducing the host cell containing the plurality of recombinant vectors into a host organism, wherein the plurality of recombinant vectors is capable of producing foamyvirus particles after introduction into the host cell. The plurality of recombinant vectors may be any embodiment of the plurality of vectors described herein. Additionally, the host cell may be any acceptable human or non-human mammalian cell, as described herein. In at least one embodiment, the plurality of recombinant vectors are operable to compensate for a genetic defect. In an exemplary embodiment, the genetic defect results in Fanconi Anemia in a host organism.

In at least one embodiment, a specific ratio of the four vectors encoding the necessary components to form FV may increase the production of infectious particles. In at least one embodiment, generation of the most infectious FV particles was produced when the ratio of the components was vector>>gag>pol>env. The level infectious particles produced through use of an embodiment of the plurality of vectors may be at least 5-times, at least 10-times, at least 20-times, at least 40-times, at least 60-times, at least 80-times, or at least 100-times the level of infectious particles produced by infection of a native foamyvirus particle into an identical cell type.

In at least one embodiment, vectors containing a human optimized expression sequence may be used to genetically modify cells. These cells may be modified so as to express a reporter system, such as EGFP, or to correct for an identified condition. In at least one embodiment, FV vectors may be used to correct or compensate for a sensitivity produced by a condition. At least one embodiment of the method of correction uses FV vectors expressing human FANCG cDNA to correct fancg−/− cells for sensitivity towards DNA crosslinking agents, such as mitomycin C. Additionally, in at least one embodiment, the above correction of fancg−/− cells may correct for the outcome of the sensitivity towards a crosslinking agent, such as apoptosis.

In at least one embodiment, a method of transfection with the optimized vectors may correct for an in vivo defect. In at least one embodiment of the method, an open reading frame (ORF) encoding a compensating element is inserted onto an optimized vector. Following incorporation of the ORF, the optimized vector is inserted into cells compatible with the in vivo recipient organism. Following insertion of the optimized vector into recipient cells, the recipient cells are then transplanted into the recipient organism. In at least one embodiment, the recipient cells may be CD117+ cells or CD45.2+ cells. Further, in an additional embodiment, the recipient cells containing the optimized vector may be co-transplanted with at least one additional cell containing an additional optimized vector, or an additional cell. In at least one embodiment, the recipient organism may be a mammal. Additionally, in at least one embodiment, the recipient organism may one of a domesticated animal, farm animal, aquatic animal, or human.

### EXAMPLES

### Example 1

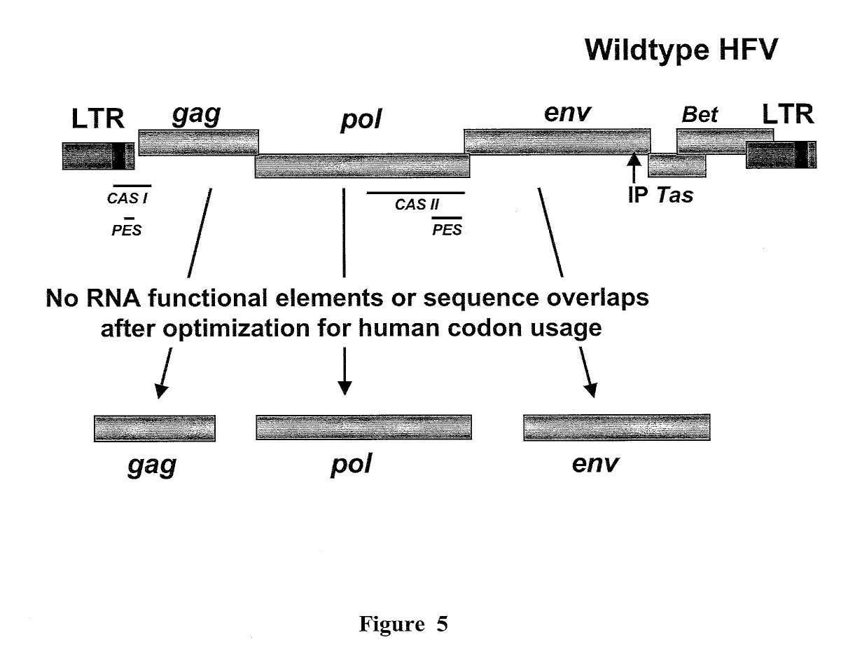

In an example of a foamyvirus expression system, domain/functional elements (CAS I, CAS II, PES I, PES II), and the overlap between the FV gag, pol and env genes were eliminated. Redundancies within the genetic code were also modified to adapt the nucleotide sequence of each FV gene for high expression in human cells. This process is shown schematically in FIG. 5. Codon usage of the FV gag, pol and env genes was adapted to the codon bias of Homo sapiens genes. Modification of regions of very high (>80%) or very low (<30%) GC content in these genes were avoided where possible. In addition, during the optimization process the following cis-acting sequence motifs were avoided, including: (1) internal TATA-boxes, chi-sites and ribosomal entry sites, (1) AT-rich or GC-rich sequence stretches, (3) ARE, INS, CRS sequence elements, (4) repeat sequences and RNA secondary structures, and (5) (cryptic) splice donor and acceptor sites, branch points.

After modification, each resulting cDNA was cloned in an expression vector containing the cytomegalovirus (CMV) promotor and corresponding splice donor (SD) and acceptor (SA) sites. Splicing with binding of snRNAs and the spliceosome complex is known to stabilize mRNAs and mark these transcripts for nuclear export. Therefore, introducing a strong promotor that gives rise to spliced transcripts enabled us to achieve maximum expression of the human codon usage-optimized FV gag, poi and env.

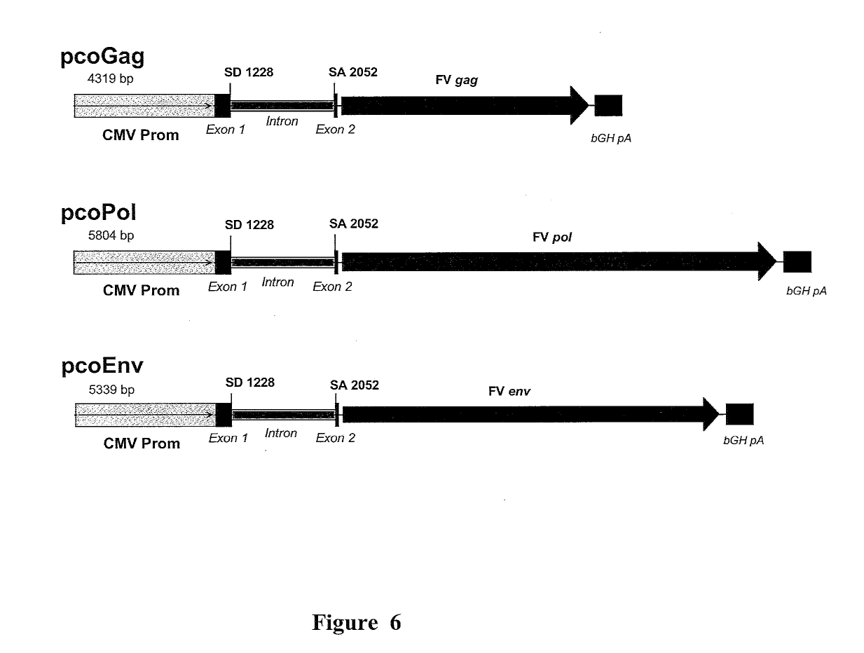

The structure of the three final packaging constructs containing the human codon optimized FV gag, pol and env genes is shown in FIG. 6.

### Example 2

To assessing the amount of protein that was present in virus producing cells upon transfection of the human codon usage-optimized cDNAs in comparison with the wild-type sequences, 293T packaging cells were transfected with equal amounts of the corresponding plasmids in sequential dilutions. DNA (pUC19) from a cloning vector was utilized to keep the absolute amount of DNA transfected into 293T cells the same. For FV gag and pol, the only difference between the two plasmids was the optimization of the FV gene for human codon usage. For the PFV env gene, the human optimized ORF in a vector with slice donor and acceptor sites was compared to the non-optimzed sequence expressed without splice sites in the same vector. Additionally, the expression of optimized simian FV (SFV) env contained on pUC19, like the above comparisons, was compared to a non-optimized SFV env contained on a pCL (Promega).

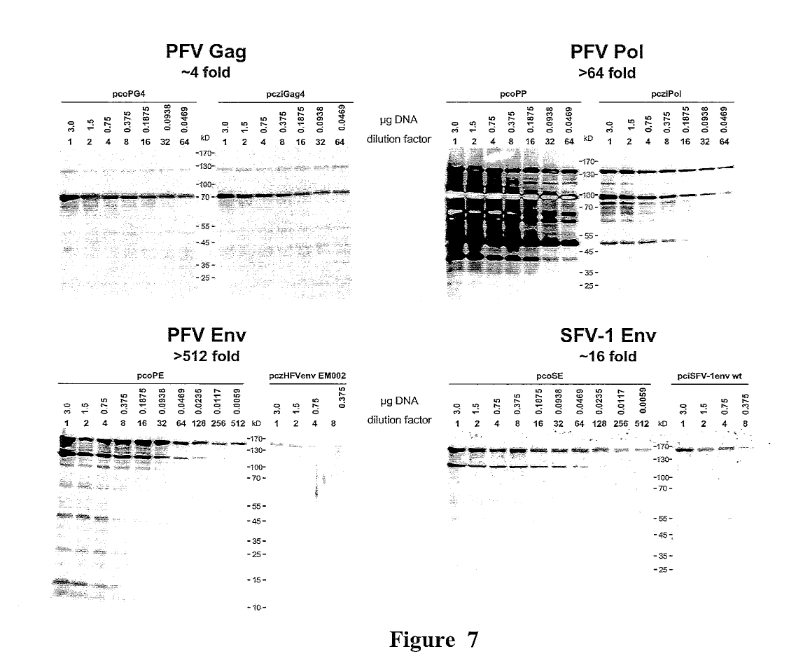

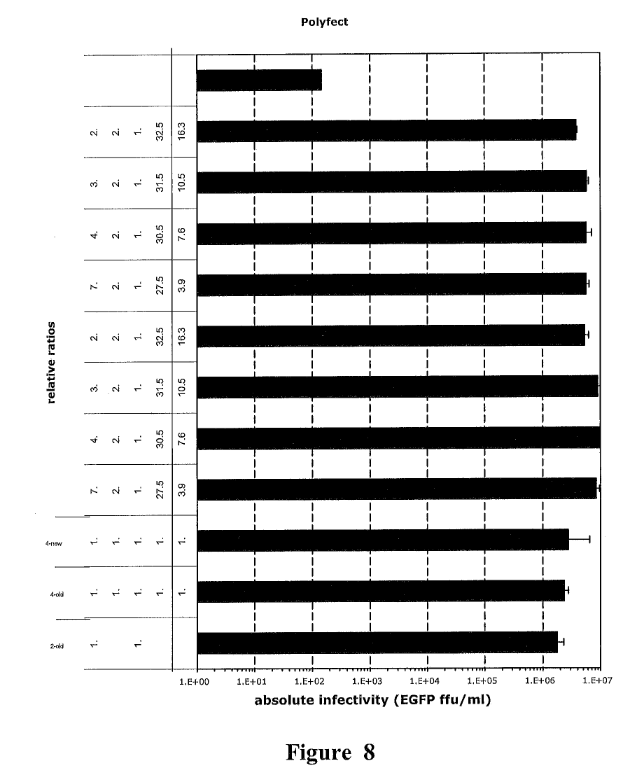

As shown in FIG. 7 for the PFV gag and pol and also for the SFV-1 env, protein levels increased between 4 and 64 fold (gag and pol) just through optimization of the FV genes for human codon usage. By combining the optimized expression construct with the splice cassette, the PFV env protein expression in 293T increased by more than 500fold compared to the non-optimized non-spliced cDNA when expressed in the otherwise identical vector from the same promotor.

### Example 3

To determine the ratio of the four plasmids for high-titer production of infectious FV particles, several concentrations of the four plasmids were tested in combinations and the stuffer plasmid used to transfect the same amount of total DNA into 293T cells. Virus was harvested from 12-well plates. After 3 days, read-out was performed by flow cytometry and assessed the EGFP expression of human fibrosarcoma cells, HT1080, exposed to limiting dilutions of FV supernatants.

From the above shown initial experiment and as well as others (not shown), maximal numbers of infectious particles were found when the concentrations of vector to gag to pol to env were used in a decreasing concentration. Therefore, in an example, the gag to pol ratio was set to 5:1 and the amount of vector and env co-transfected into 293T cells in a 10 cm dish using active dendrimers (polyfect@) as transfection reagent was varied. Read-out was by way of EGFP expression of HT1080 cells after serial dilutions of the FV supernatants.

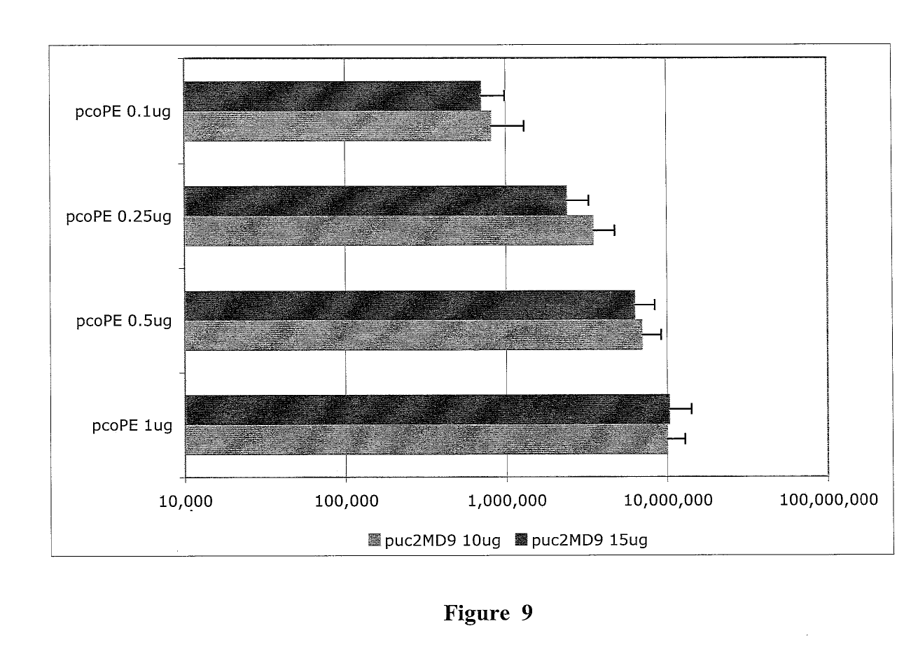

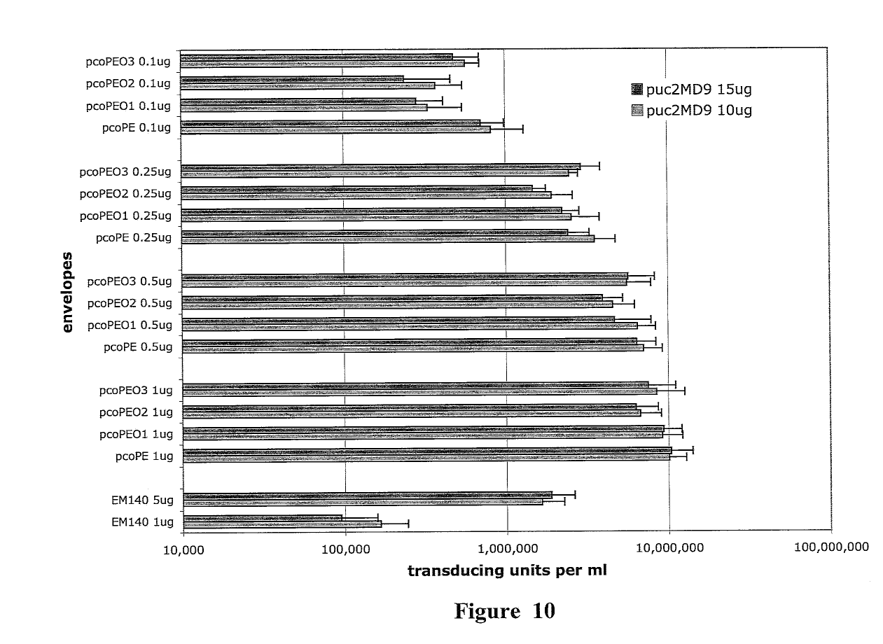

These results demonstrated that with a concentration of 5 μg gag and 1 μg pol plasmid per 10 cm dish of 293T cells, there is no increase in the production of infectious FV particles when comparing 15 and 10 μg of vector plasmid (FIG. 9, 10). Therefore, the vector to gag to pol plasmid ratios were set at 10:5:1. The codon optimization and improved expression of PFV env however had a clear effect, a two log increase, on the amount of infectious particles, when comparing the pcoPEO1 with the pcEM0140 plasmid at 1 μg plasmid/dish (FIG. 10). There was no difference however between various env mutants for the production efficiency of recombinant foamyviruses (FIG. 9, 10).

### Example 4

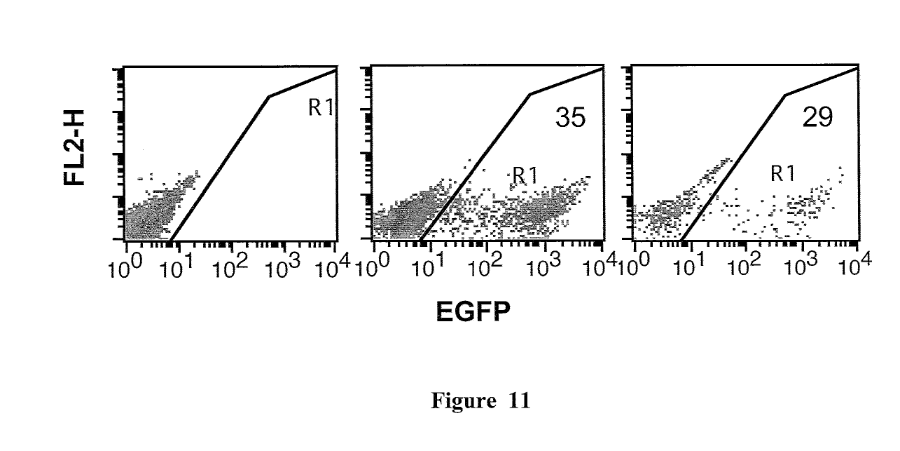

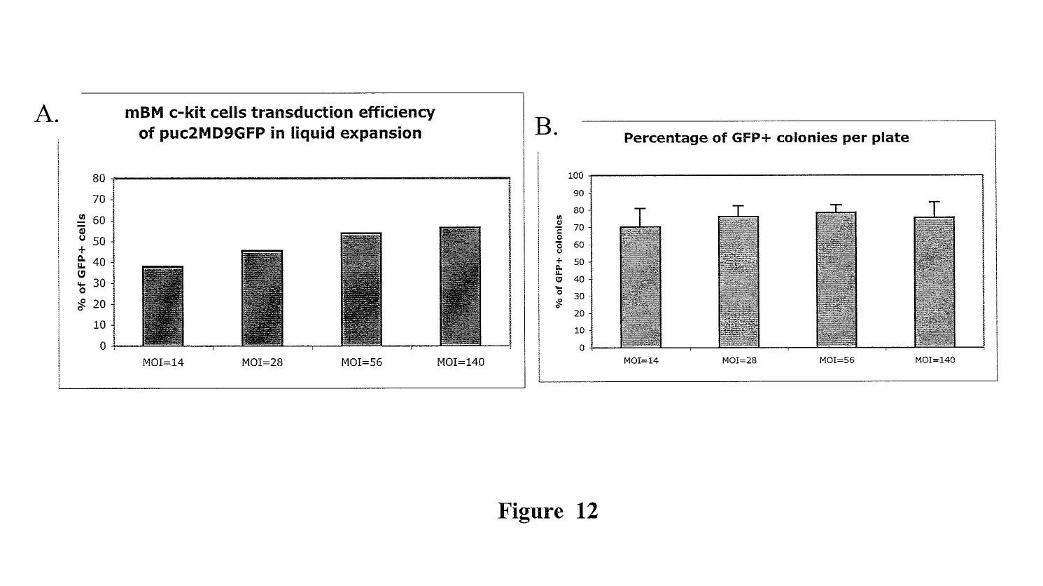

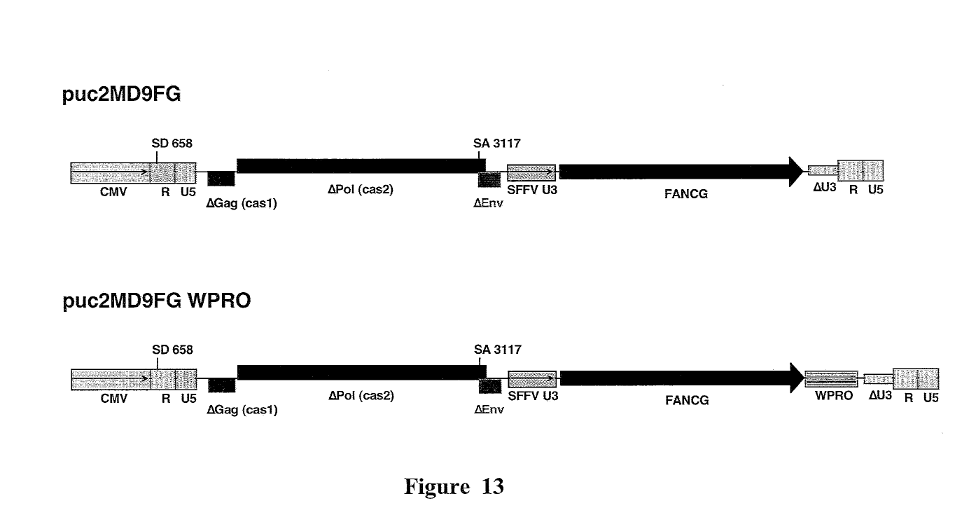

To determination of whether the improved production of infectious FV particles was instrumental to transduction efficiently primary murine and human cells, murine mononuclear cells were harvested from the bone marrow of WT C57/B16 mice or of mice with a targeted disruption of the murine Fancg gene. CD117 positive cells were isolated by MACS and then transduced overnight on the fibronectin fragment CH-296 at different multiplicities of infections (MOIs) with a FV vector, MD9, that expresses EGFP off an internal SFFV promotor. After 14 hours, transduced cells were placed in standard progenitor assay or expanded for 5 days in medium supplemented with cytokines. Cells from the liquid expansion were analyzed by flow cytometry using EGFP expression as readout for gene transfer efficiency (FIG. 11). Progenitor assays were counted after 7 days for the % of cells expressing EGFP. These results showed that the optimized FV production system could readily be used to genetically modify murine WT (not shown) and Fancg−/− hematopoietic cells.

### Example 5

FV vectors expressing the human FANCG cDNA were analyzed to determine whether they were able to correct murine Fancg−/− cells from their characteristic sensitivity against the DNA cross-linking agent mitomycin C (MMC). In these studies, the human FANCG cDNA was placed under control of the internal SFFV promotor in the FV MD9 vector, either with or without an optimized woodchuck hepatitis virus post-transcriptional regulatory element (WPRO) for mRNA stabilization.

Recombinant FV particles were produced using the 3 optimized expression plasmids for gag, pol and env. CD117+ cells from WT and Fancg−/− mice were transduced at an MOI of 10 based on the EGFP expression in HT1080 indicator cells and plated in standard progenitor assays with increasing concentrations of MMC. Results are shown as % of colony formation of clonogenic cells not exposed to MMC.

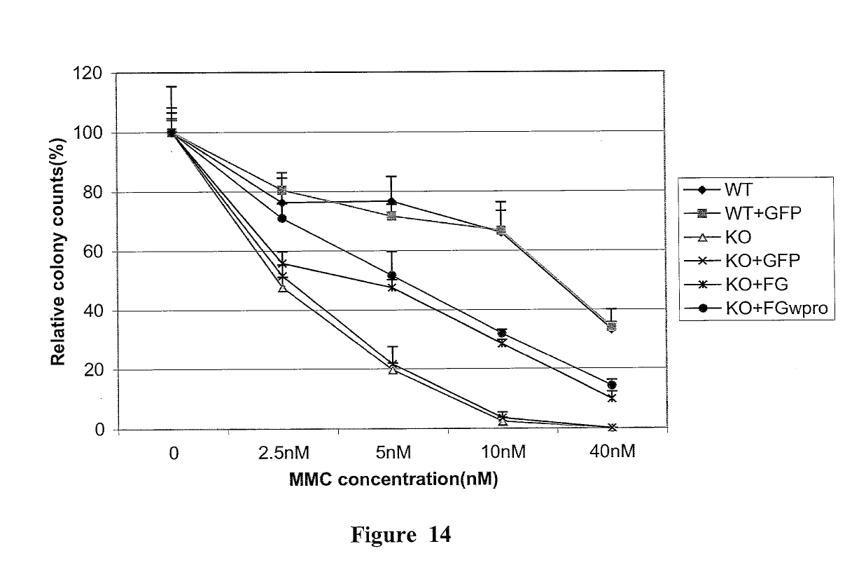

The gene transfer of cells in the experiment shown in FIG. 14 was around 30% as judged by the amount of EGFP+ cells in the in vitro assays. As the human FANCG cDNA is almost triple the size of EGFP, more than 30% of the Fancg−/− cells were corrected after transduction. Therefore, the percentage of correction as assessed by the survival of clonogenic cells in the presence of MMC (FIG. 14) suggests that both FV vectors expressing FANCG were capable of completely correcting the germ-line mutations in these primitive murine cells.

### Example 6

Additionally, two FANCG cDNA expressing MD9 vectors were determined to be capable of correcting the cellular phenotype of primary human FA-G fibroblasts when challenged by incubation with MMC. To this end, the FV vectors carrying either the FANCG or the EGFP cDNAs were produced with the human codon optimized FV packaging system and then utilized to transduce FANCG-deficient human cells.

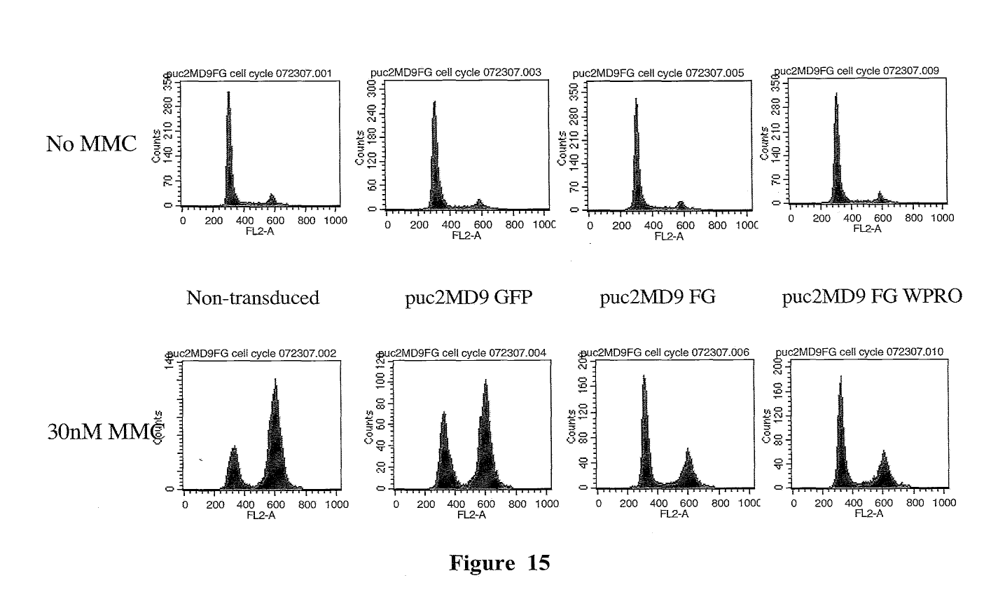

As shown in FIG. 15, MMC induced the typical cell arrest 72 hours after exposure to the bi-functional cross-linking agent in untransduced cells and in cells transduced with the EGFP control vector. In contrast, SFFV driven FANCG expression from both MD9 vectors was sufficient to correct the cellular FA phenotype in these patient derived cells.

### Example 7

In an additional example, the optimized FV production system was evaluated to determine whether it was instrumental for transducing murine hematopoietic stem cells and thereby correcting the repopulation defect as observed in murine Fancg−/− cells in competitive repopulation assays. To this end, FV MD9 vectors expressing either the FANCG or the EGFP cDNA were produced with the optimized packaging system and the supernatants then utilized to transduce WT or Fancg−/− CD117+ cells obtained from the bone marrow of mice. 14 h after exposure to the FV vectors, transduced cells were co-transplanted with WT CD45.1+ competitor cells into lethally irradiated recipients. Gene transfer efficiency into CD45.2+ test cells was assessed for the control vector by EGFP protein expression in CD45.2+ cells as determined by FACS analysis. Shown are EGFP+ CD45.2+ cells 6 month after trans-plantation in the PB of a mouse.

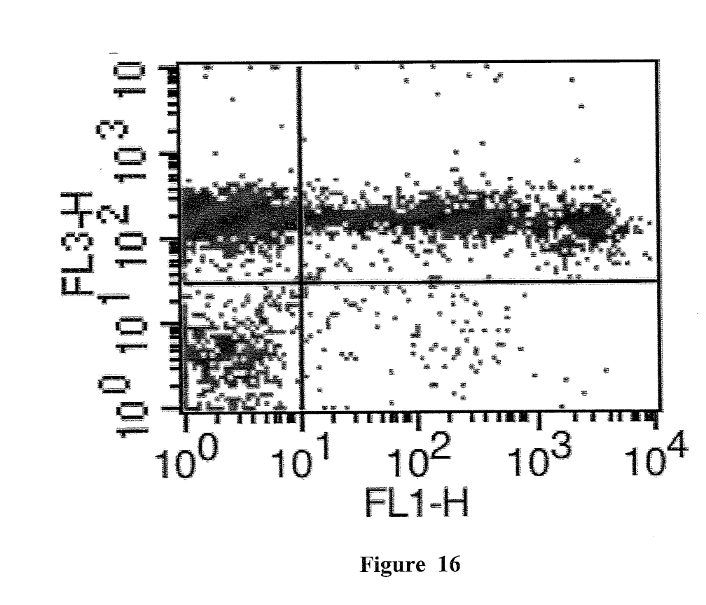

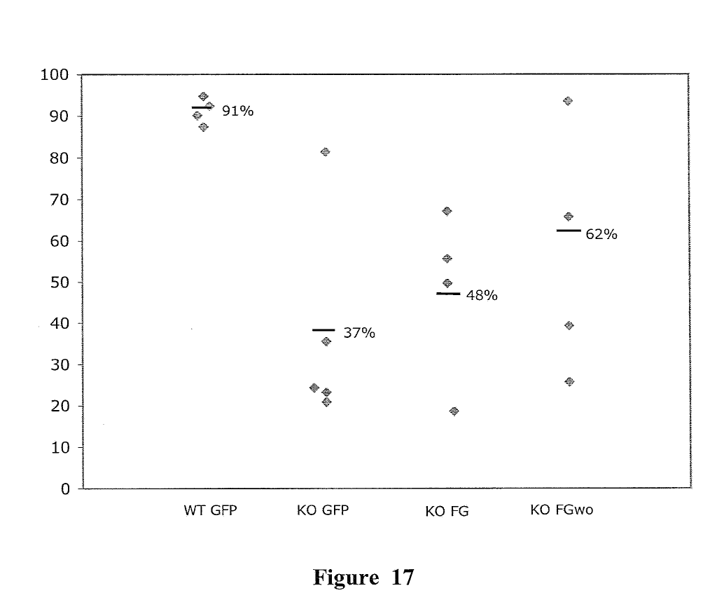

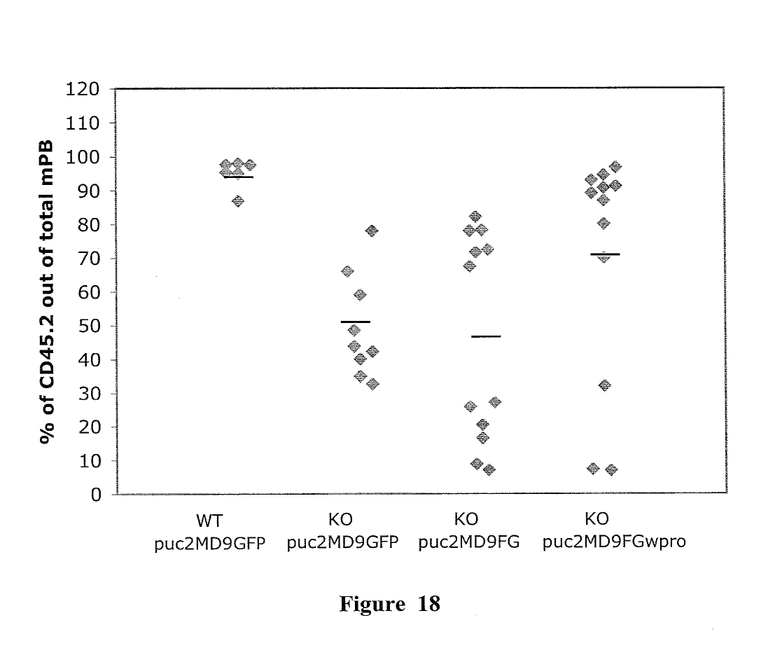

Successful correction of the repopulation defect of Fancg−/− CD45.2 stem cells through expression of the human FANCG protein was assessed 6 months after transplantation by analyzing the percentage of CD45.1 and CD45.2 cells in the PB of reciepient mice. As shown in FIG. 16, the repopulation defect of CD45.2+ test cells was clearly detectable when compared to the CD45.2 WT cells both transduced with the EGFP control vector. Expression of the human FANCG increased the % of CD45.2+ cells in the PB when compared to the transduced noncorrected KO EGFP cells.

To prove that hematopoietic stem cells were genetically modified by the FV vectors, mice were sacrificed after 8 months and BM cells from each mouse injected into three lethally irradiated secondary recipients, Chimerism analyses 2 months after transplantation demonstrated the improved repopulating ability was maintained for approximately half of the mice who received cells transduced with the FANCG transgene. Due to the relatively low MOI, it is assumed that not all mice in this group had received corrected stem cells. For the EGFP mice, the level of EGFP positive cells in the PB were similar between the first and the secondary transplantations.

### Example 8

### Materials and Methods

### Mice

Fancc−/− and Fancc+/+ mice (C57B1/6xSV129) were backcrossed 10 generations into /a C57B1/6 stain (CD45.2+). Congenic C57B1/6 strain (CD45.2+) and B6.SJLPtracpep3b/BoyJ (BoyJ) mice (CD45.1+) were originally purchased from Jackson Laboratories (Bar Harbor, Me.) and are maintained in our animal facility.

### Foamyviral Vectors and Virus Production.

The foamyviral constructs used in these studies were derivatives of the MD9 construct. In the MD9 vector, all foamyviral genes and also the enhancer elements in the 3′ U3 region (FIG. 1) have been functionally inactivated by partial deletions. The remaining noncoding 5′ region of GAG and the 3′ region of POL are harboring the packaging signals (CAS I and II) and are therefore used for the production of recombinant foamyviral particles. A linker was cloned into the Not I site 3′ of MD9 and then an expression cassette containing the encephalomyelocarditis virus (EMCV) internal ribosomal entry site (IRES) and the enhanced green fluorescence protein (EGFP) cDNAs introduced from S11IEG3 via BamH I and Spe I, thereby creating the MD9-EGFP construct. The human FANCC cDNA was cloned into the

BamH I site resulting in the MD9-FANCC/EGFP vector only. FV-containing supernatant was generated in 293T cells with the FV helper plasmid pcgpl and the FV envelope plasmid EMO2 as previously described. The titers of the viral supernatant were 1-5×10⁷ viral particles/ml for MD9-EGFP and 4-10×10⁶ viral particles/ml for MD9-FANCC/EGFP construct after concentration.

### Foamyvirus-Mediated BM Transduction and Transplantation

BM cells were obtained from Fancc−/− or Fancc+/+ wildtype (WT) mice and purified for c-kit/CD117 positive cells, as described previously. CD117+ Fancc−/− cells were transduced for 14 hours with the foamyviral vectors (MOI 20) on non-tissue culture treated plates treated with the recombinant human fibronectin fragment CH296, RetroNectin™ (2 μg/cm2, TAKARA BIO INC, Otsu, Japan) as previously described in the presence of mIL-6 (200 U/mL) and mSCF (100 ng/mL; both from Peprotech, Rocky Hill, N.J.). WT cells were transduced with MD9-EGFP only. Cells were harvested following transduction, washed twice with 10-15 volumes of phosphate buffered saline (PBS) and 5×10⁵ of transduced CD117+ cells injected into the tail vein of lethally irradiated 8 to 10 weeks old C57B1/6 WT recipients, as described previously. In parallel, an aliquot of the transduced cells were plated in semisolid medium that promotes clonogenic growth of myeloid progenitors to determine the transduction efficiency.

### FANCD2 Western Blot

Unaffected human or FANCC deficient fibroblasts (GM3136) were cultured in Iscove's modified Dulbecco's medium (IMDM) containing 15% fetal calf serum and 1% penicillin G and streptomycin. They were transduced with foamy viral vectors encoding either the reporter gene only or MD9-FANCC/EGFP. 48 hrs following transduction, the cells were treated with IR (1000 Rad) and 3 hrs later protein extracts were isolated. 20 μg of protein extract was analyzed by Western blotting using a monoclonal antibody (1 μg/ml; BD Biosciences, San Jose, Calif.) that recognizes FANCD2 and monoubiquitinated FANCD2. The protein was detected with an anti-mouse antibody conjugated to horseradish peroxidase and developed using the ECL-Plus system (Amersham Biosciences, Piscataway, N.J.).

### Transplantation Protocols for Competitive Repopulation Assay

Transduced, washed CD117+ cells were mixed with a common pool of BoyJ mononuclear cell (MNC) competitors and transplanted into 8 to 10 week old lethally irradiated C57B1/6 mice as described. In cohort 1, 1.5 ×10⁵ test cells were transplanted with 5×10⁵ competitor cells. In cohort 2, twice as many test cells were administered to recipients transplanted with Fancc−/− cells transduced with the virus encoding EGFP only as in the other experimental groups in an attempt to equalize chimerism. Mean donor chimerism was analyzed to evaluate for significant differences between groups. CD45.1 and CD45.2 chimerism were analyzed monthly following transplantation as previously described. Repopulating units (RU) were calculated as (competitor numbers×10⁵×% donor chimerism)/ (100−% donor chimerism) as described previously. An unpaired Student's t-test was used to determine whether significant differences existed in chimerism between genotypes only.

### Hematopoietic Progenitor Assays and Drug Resistance

FV-transduced cells were plated in triplicate 35-mm plates (Becton Dickinson, Franklin Lakes, N.J.) with increasing concentration of MMC or tumor necrosis factor alpha (TNF-α) as described. To determine the phenotypic correction or the gene transfer efficiency, the number of total colonies formed per plate was enumerated and the EGFP expressing colonies were counted by the fluorescent microscopy.

### Amplification of Genomic and Proviral DNA

Colonies of FV-transduced cells plated in progenitor assays were individually collected and suspended in PBS. The genomic DNA was isolated and polymerase chain reaction (PCR) for EGFP was performed: Forward 5′-ATGGTGAGCAAGGGCGAGGAG-3′, Reverse 5′-AAGTCGTGCTGCTTCATGTG-3′ with the following program: 95° C., 5 minutes; 95° C., 40 seconds; 55° C., 30 seconds; 72° C., 1 minute; cycled to step 2 for 31 cycles; 72° C., 10 minutes and then stored at 4° C. and analyzed on a 1% agarose gel. The amplified product has a size of approximately 250 bp. In addition, PCR for the genotype of the progenitor cells was performed. Three primers are used: 5′-GAGCAACACAAATGGTAAGG-3′, 5′-CCTGCCATCTTCAGAATTGT-'3 and 5′-TTGAATGGAAGGATTGGAGC-3′ with the following program: 95° C., 5 minutes; 95° C., 30 seconds; 55° C., 2 minutes; 72° C., 1.5 minutes; cycled to step 2 for 31 cycles; 72° C., 10 minutes and then stored at 4° C. and analyzed on a 1% agarose gel. The amplified product of the WT copy of the Fancc gene is approximately 800 bp, whereas the knockout gene PCR product is approximately 600 bp.

### Ligation-Mediated Polymerase Chain Reaction (LM-PCR)

For detection of FV integration sites, BM MNC from two primary recipients of Fancc−/− mice transduced with MD9-FANCC/EGFP from the first cohort and two primary recipients from the second cohort were enriched for CD45.2 positive cells by FACS and then plated in standard progenitor assay. From each of the four mice, 20 progenitor colonies were picked and then subjected to LM-PCR as described previously with minor variations. The restriction enzyme used was HaeIII (New England Biolabs, Frankfurt, Germany). The biotinylated primer 5′biotin-GTACAATCTAGGTGACCACTTTC-3′ (407) was used in a one step extension at 94° C., 15 minutes; 58° C., 2 minutes; 72° C., 10 minutes; 2 cycles. The two internal primers for the nested PCR were 5′-TCTCATCCCAGGTACGTCTATGA-3′ (404) and AP2 as previously described. The DNA from excised bands was cloned into pCR2.1 using the TOPO cloning kit (Invitrogen) and then sequenced on an ABI Gene Amp 3770 System. As described previously, SeqMap (http://seqmap.compbio.iupui.edu/) was used to map the sequences against the mouse genome. This was then confirmed by mapping the positions using the ENSEMBL website (http://www.ensembl.org/) and mus musculus database release, December 2006 and the UCSC Genome Browser (http://genome.ucsc.edu).

### Southern Blot for Junctional Fragment Analysis

Genomic DNA from BM and spleen specimens was isolated using phenol-chloroform extraction and digested with XhoI (New England BioLabs, Ipswich, Mass.). Fragments were isolated via ethanol precipitation and run on a 1% agarose gel. The DNA was only transferred to a nylon membrane using the TurboBlotter™ system (Schleicher & Schuell, Keene, N.H.). To generate the hybridization probe, the MD9 plasmid was digested with PstI (New England BioLabs, Ipswich, Mass.) and the 1655 by fragment was isolated using QlAquick gel extraction kit (Qiagen, Valencia, Calif.), labeled using the Prime-It II Random Primer labeling kit

(Stratagene, La Jolla, Calif.) and purified using a micro-spin 30 column (Bio-Rad, Hercules, CA). The membrane was pre-hybridized for 2 hrs at 42° C. with the hybridization solution (6×SSC, 50% Formamide, 5× Denhardt's, 0.5% SDS in water) supplemented with 100 μg/ml denatured salmon sperm DNA (Stratagene, La Jolla, Calif.). After pre-hybridization, the membrane was hybridized for 16 hrs at 42° C. with the hybridization solution supplemented with 100 μg/ml denatured salmon sperm DNA (Stratagene, La Jolla, Calif.) and denatured labeled probe. The next day, the membrane was washed four times for 15 minutes at 42° C. with the wash solution (2× SSC, 0.1% SDS in water) and exposed to film (BioMax MS film Kodak, Rochester, N.Y.) at −80° C. with a Cronex Lightning Plus intensifying screen (DuPont, Wilmington, Del.).

### Example 9

Efficient transduction of hematopoietic progenitors from Fancc−/− mice by short-term exposure to FV vectors in the absence of pre-stimulation.

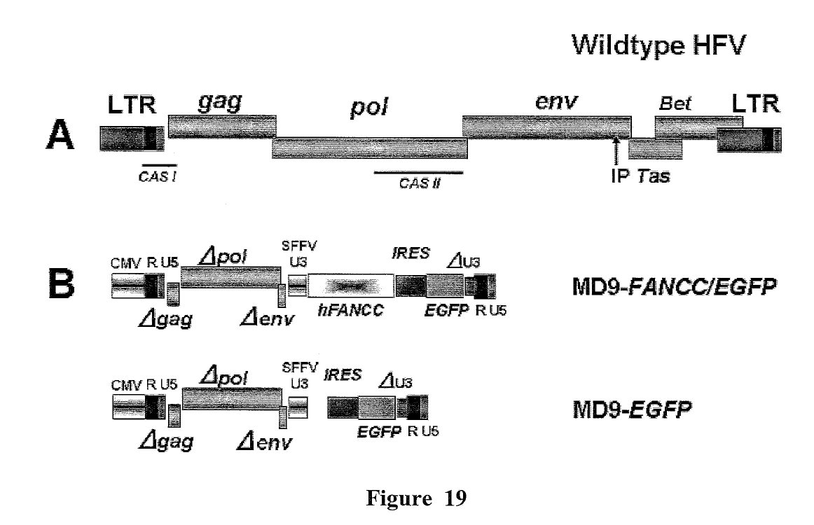

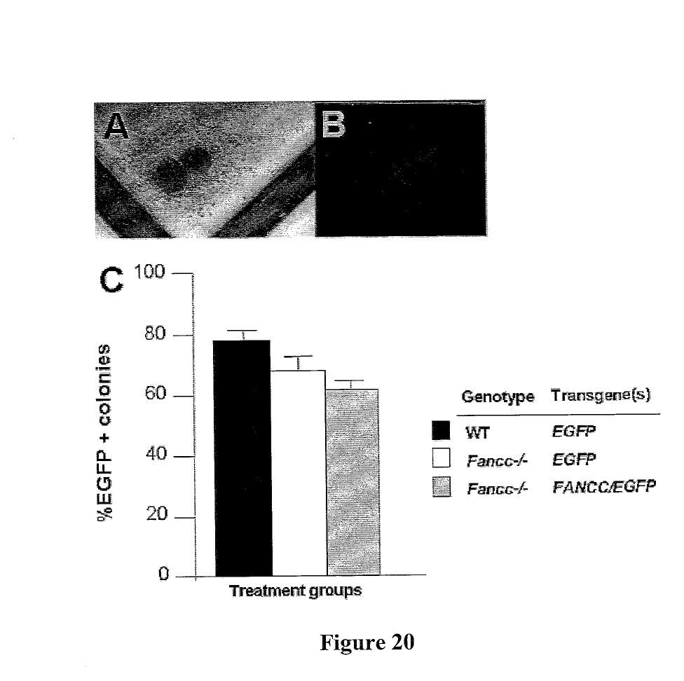

The recombinant MD9 FV vector was utilized (FIG. 19) to express the human FANCC and EGFP cDNA linked via an EMCV IRES element. This expression cassette was under the transcriptional control of the spleen focus forming virus (SFFV) promoter element, which had been sufficient to mediate expression of transgenes in NOD/SCID repopulating human CD34+ umbilical cord blood cells, An MD9 vector that expresses the EGFP transgene only was used as a control (FIG. 19). CD117+ cells from WT and Fancc−/− mice were transduced with foamyviral vectors in the presence of growth factors and viral supernatants on CH296-coated plates for 14 hrs. Recovery of CD117+ cells after the overnight transduction protocol was comparable in all experimental groups and was consistently 70-80% of input cells (data not shown). The next day, cells were cultured in semisolid medium to determine the gene transfer efficiency into clonogenic myeloid progenitors. After 7 days, the proportion of EGFP positive myeloid progenitors was scored (FIG. 20). Collectively, short-term exposure of both WT and Fancc−/− CD117+ cells to FV supernatants on recombinant fibronectin resulted in a gene transfer efficiency of greater than 50% of clonogenic progeny based on EGFP expression in 4 independent experiments.

### Example 10

Foamy virus mediated expression of FANCC corrects the DNA damage and inflammatory cytokine hypersensitivity of Fancc−/− myeloid progenitors.

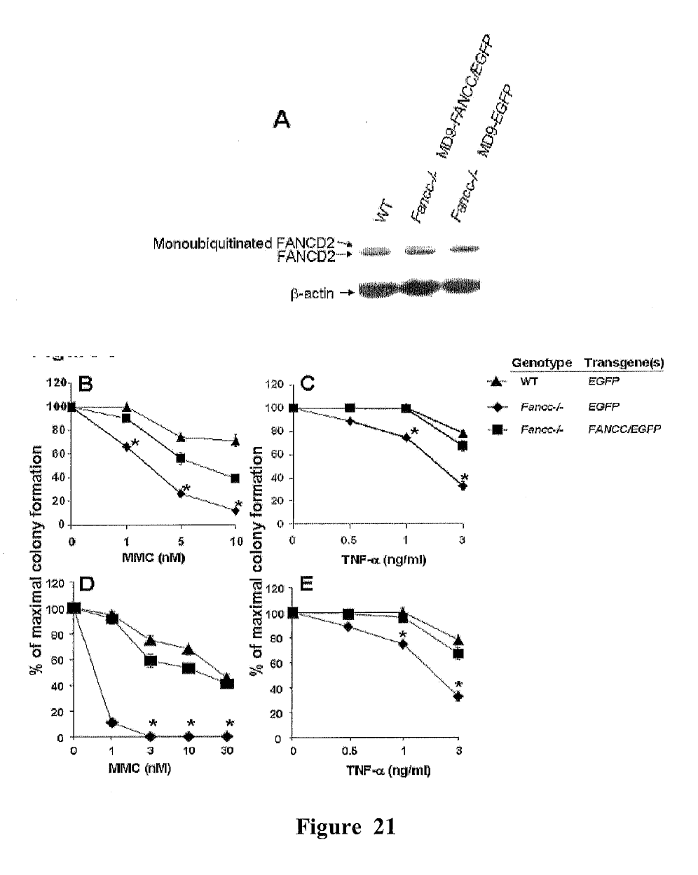

Since FANCC is a component of the FA core nuclear complex and is required for FANCD2 monoubiquitination in response to DNA damage and during S-phase, the detection of monoubiquitinated FANCD2 can be used as a measure of functional FANCC protein. Unaffected human cutaneous and FANCC deficient fibroblast lines were transduced and 48 hrs later the cells were treated with 1000 Rads of ionizing radiation. Three hours subsequently, cells were harvested and protein extracts were isolated. As expected, the FANCC deficient fibroblasts transduced with the MD9-EGFP reporter construct do not express the monoubiquitinated form of FANCD2 (FIG. 21A).

In contrast, FANCC deficient fibroblasts transduced with MD9-FANCC/EGFP can restore the assembly of the FA nuclear complex, leading to efficient monoubiquitination of FANCD2 (FIG. 21A). These data provide biochemical evidence indicating that the FV vector encoding human FANCC expresses a functional recombinant protein that is sufficient to allow activation of the downstream effector FANCD2. To determine whether the SFFV promoter mediated expression of FANCC was sufficient to phenotypically correct Fancc−/− primitive clonogenic cells, CD117+ cells were transduced with MD9-FANCC/EGFP or the reporter construct and clonogenic assays of myeloid progenitors were established in the presence of a range of concentrations of MMC, a bifunctional alkylating agent, or the inhibitory cytokine TNF-α Consistent with established work, Fancc−/− progenitors transduced with the reporter construct were hypersensitive to both MMC and TNF-α. In contrast, progenitors transduced with the construct expressing FANCC were corrected to WT levels (FIG. 21B and 21C). As an initial assessment of in vivo function of the expressed FANCC protein, transduced CD117+ cells were transplanted into lethally irradiated syngeneic recipients to allow long-term reconstitution of the hematopoietic system by genetically modified stem cells. Six months after transplantation, BM low-density MNC from the reconstituted mice were isolated and clonogenic cells from the respective experimental groups were cultured in a range of concentrations of MMC or the inhibitory cytokine TNF-α. Consistent with previous studies, and with the clonogenic assays conducted prior to transplantation (FIG. 21B and 21C), the Fancc−/− progenitors transduced with the reporter construct were hypersensitive to both MMC and TNF-α (FIG. 21D and 21E). In contrast, progenitors isolated from mice reconstituted with Fancc−/− cells expressing the FANCC transgene had sensitivities to MMC and TNF-u that were comparable to progenitors from WT mice (FIGS. 21D and 21E). These findings support the hypothesis that FV expression of human FANCC introduced via this short-term transduction protocol is sufficient for transduction and functional complementation of Fancc−/− longterm repopulating cells to cytotoxic agents.

### Example 11

Foamyviral transfer of FANCC restores the repopulating ability of Fancc−/− stem cells to WT levels.

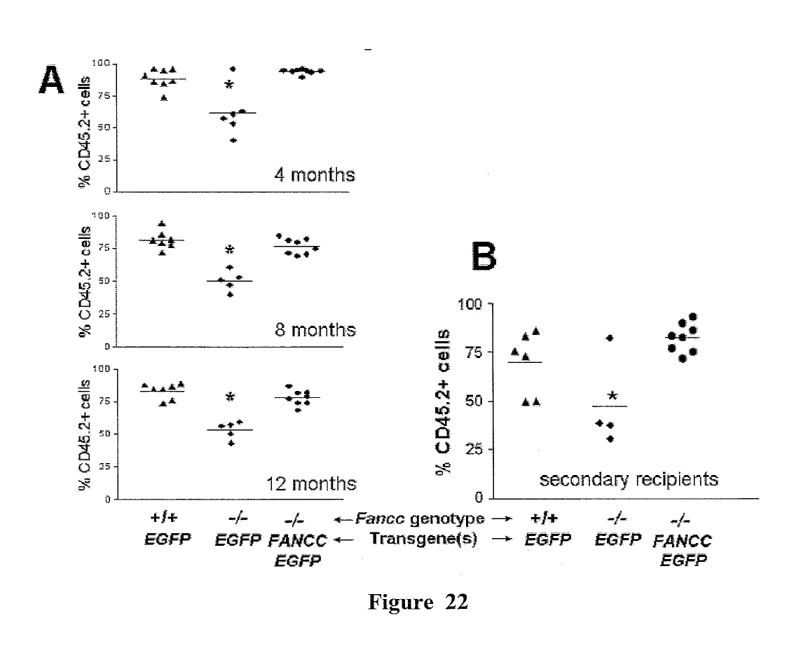

Competitive repopulation is an established quantitative measure of stem cell repopulating activity that allows a direct comparison of the proliferative activity of reconstituted stem cells of different genotypes via their relative proliferation of myeloid and lymphoid lineages to a common pool of competitor cells. Since murine Fancc−/− BM cells have reduced repopulating ability compared with WT cells and a prevalent phenotype in FA patients is BM failure, this methodology was used to assess the potential of the foamyviral vector MD9-FANCC/EGFP to correct the repopulating ability of Fancc−/− stem cells to that of syngeneic WT stem cells. CD117+ WT and Fancc−/− BM cells were isolated, transduced with either the MD9-FANCC/EGFP or the control vector and then co-transplanted with a common pool of CD45.1+ competitors into lethally irradiated recipients using previously established methods. As shown in FIG. 22A, the test cell chimerism of individual recipients from one of two independent experiments with 6-8 primary recipients per experimental group was followed on serial measurements over the course of one year. Consistent with previous results, recipients reconstituted with mock-transduced Fancc−/− cells have a reduced repopulating ability compared to recipients reconstituted with WT cells. In contrast, mice reconstituted with Fancc−/− cells expressing the FANCC cDNA after foamyviral gene transfer have a test cell chimerism that is comparable to recipients transplanted with WT cells at all time points measured (FIG. 22A). At sacrifice, the repopulating activities of the respective test cell populations were assessed by measuring the number of repopulating units (RU) as originally described by Harrison. The results demonstrate that the repopulating ability of Fancc−/− cells expressing the EGFP reporter transgene are only approximately 17% of WT marrow, while the RU of Fancc−/− cells expressing the recombinant FANCC transgene were comparable to WT marrow (Table I). Comparable results were obtained from a second independent experiment also containing 6-8 recipients in each experimental group (data not shown). The improvement of the repopulating ability in Fancc−/− cells transduced with MD9-FANCC/EGFP construct as compared to the activity of WT test cells supports the hypothesis that foamyviral expression of the transgene is correcting the repopulating activity of Fancc−/− stem cells.

TABLE I
Repopulating units 12 months post transplantation

| Fancc | | Number of | Repopulating |
|---|---|---|---|
| genotype | Vector | mice (n) | units |
| +/+ | MD9-EGFP | 7 | 6.11 ± 0.96 |
| −/− | MD9-EGFP | 5 | 1.03 ± 0.11* |
| −/− | MD9- | 8 | 7.01 ± 0.95 |
| | FANCC/EGFP | | |

Quantities shown are mean ± SEM.
*P < 0.05 compared to either WT MD9-EGFP or Fancc−/− MD9-FANCC/EGFP

To confirm that the correction in repopulating ability is associated with transduction of the transgene, CD45.2+ BM cells were sorted by FACS and plated in standard progenitor assays. DNA from individual progenitors was isolated and amplified to assess the genotype and the presence of the transgene. The PCR analysis of 70 colonies from 7 recipients containing Fancc−/− MD9- FANCC/EGFP test cells demonstrated that 67/70 (96%) of the colonies were positive for the Fancc−/− genotype. A representative analysis is shown in FIG. 22C. A high proportion of those same Fancc−/− test progenitors (90%) also contained the provirus. A representative blot demonstrating this is shown in FIG. 22D. The majority of progenitors (25/29) analyzed from CD45.2+ bone marrow cells of three representative recipients transplanted with Fancc−/− cells transduced with MD9-EGFP also were Fancc−/−. Given the stochastic nature of the hematopoietic system, the high proportion of Fancc−/− progenitors that contain the MD9-FANCC/EGFP provirus, and the 6-7 fold difference in repopulating activity observed in Fancc−/− test cells containing the provirus encoding FANCC/EGFP vs EGFP only, we conclude that the test cell chimerism in recipients who received Fancc−/− cells transduced with the MD9-FANCC/EGFP provirus reflects the functional correction of the FA pathway.

To assess that the transgene was transduced into a stem cell with repopulating ability as well as the long term proliferative ability assessed in primary recipients, low-density MNC from selected primary recipients were transplanted into lethally irradiated recipients. The chimerism 6 months following transplantation into secondary recipients did not change and the test cell chimerism of mice reconstituted with corrected Fancc−/− cells remained comparable to that of recipients transplanted with WT cells (FIG. 22B) indicating that the transgene was functional in repopulating stem cells. In that same experiment, recipients reconstituted with Fancc−/− cells transduced with the FANCC transgene had comparable numbers of “test” colony forming units/femur (2.8±0.1×10⁴) as WT recipients (2.4±0.2×10⁴), while recipients reconstituted with Fancc−/− test cells encoding EGFP only had significantly reduced “test” progenitors (1.5±0.3×10⁴).

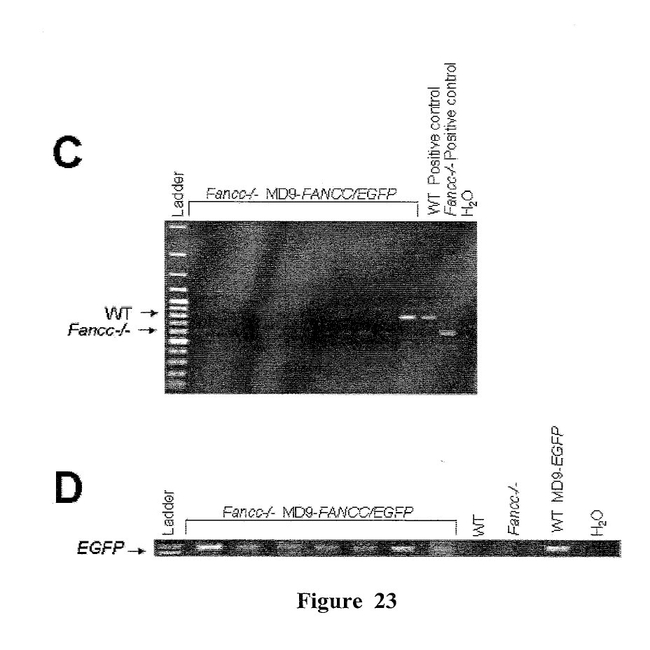

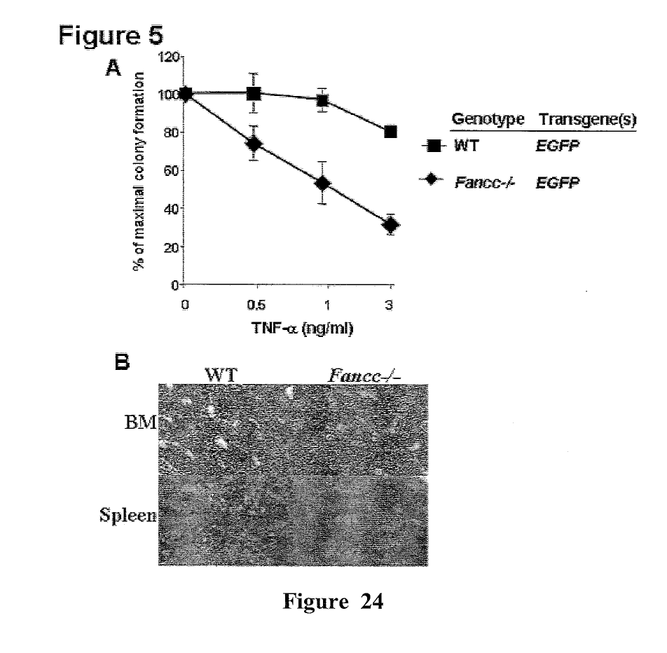

To evaluate for myelodysplasia (MDS) in mice reconstituted with Fancc−/− cells, two separate studies involving 5 cohorts of mice showed that in vitro culture of uncorrected Fancc−/− BM for 2-4 days prior to transplantation predisposes the recipients of those cells to MDS. A characteristic of the MDS phenotype observed in Fancc−/− mice is that myeloid progenitors are resistant to the apoptotic signals induced by TNF-α in progenitor assays. To determine whether this indicator of malignant transformation was present in recipients reconstituted with mock transduced Fancc−/− cells, CD45.2+ cells from primary recipients 12 months following transplantation were sorted using FACS and myeloid progenitors were cultured. Analyses revealed that the Fancc−/− progenitors retained the characteristic hypersensitivity to TNF-α (FIG. 23A). Additionally, the BM and spleen of 7 primary recipients and 4 secondary recipients reconstituted with Fancc−/− cells expressing the EGFP transgene only were examined at postmortem and had normal architecture (FIG. 23B). Similar histologic data were found in primary and secondary recipients receiving MD9-FANCC/EGFP transduced Fancc−/− cells (data not shown). Collectively, all functional and histological data fail to detect evidence of transformation of uncorrected Fancc−/− stem cells in primary or secondary recipients.

### Example 12

### Evaluation of Proviral Integrants

To assess the number of integrations that are present in progeny of the transduced Fancc−/− stem cells, BM low-density MNC from four primary recipients were harvested at the time of secondary transplants, sorted for CD45.2 expression and then plated in methylcellulose assays. After one week, 20 colonies were picked for each mouse and subjected to standard LM-PCR analysis for identifying the location of the provirus in the genome. As described previously, the position of the transgene from each colony was evaluated using the SeqMap website (http://seqmap.compbio.iupui.edu/) and confirmed using the http://genome.ucsc.edu and http://www.ensembl.org/ websites. From each mouse, one proviral integration was detected (Table II). Each integration site detected was independent of the others and was contained either in an intron or outside of a gene, consistent with other studies of FV integration patterns. Because progenitors provide limited amounts of DNA for LM-PCR analysis and proviral integrants may not always be detected, integrations were also evaluated from whole BM and spleen samples by Southern analysis in a replicate set of experiments using similar transduction conditions. Results by Southern blot revealed one junctional fragment per recipient for 9/12 mice and two junctional fragments per recipient for 3/12 mice evaluated. These data are summarized in Table III.

TABLE II
Location of observed proviral integration
sites on the mouse genome

| Mouse | Chromosome | Location | Gene ID |
|---|---|---|---|
| 91 (cohort 1) | 16 B4 | upstream of Zbtb20 | NM019778 and |
| | | and Ndufs5 | BC002163 |
| 93 (cohort 1) | 5 E3 | no nearby gene | |
| 1 (cohort 2) | 10 B3 | Intron 2 of Pkib | NM_008863 |
| 2 (cohort 2) | 13 A3.1 | no nearby gene | |

TABLE III
Proviral integration number in the mouse genome

| | | # of mice | # of mice with | # of mice with |
|---|---|---|---|---|
| Genotype | Transgene | (n) | 1 integrant | 2 integrants |
| Fancc−/− | FANCC/EGFP | 6 | 5 | 1 |
| Fancc−/− | EGFP | 3 | 1 | 2 |
| WT | EGFP | 3 | 3 | 0 |

While various embodiments of compositions, methods of production of the compositions, and methods of use of the compositions have been described in considerable detail herein, the embodiments are merely offered by way of non-limiting examples of the invention described herein. Many variations and modifications of the embodiments described herein will be apparent in light of the this disclosure. It will therefore be understood that various changes and modifications may be made, and equivalents may be substituted for elements thereof, without departing from the scope of the invention. Indeed, this disclosure is not intended to be exhaustive or to limit the scope of the invention. The scope of the invention is to be defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. It will be appreciated that other sequences of steps may be possible. Therefore, the particular order of the steps disclosed herein should not be construed as limitations on the claims. In addition, the claims directed to a method and/or process should not be limited to the performance of their steps in the order written, and it will be readily appreciated that the sequences may be varied and still remain within the spirit and scope of the present invention.

It is therefore intended that the invention will include, and this description and the appended claims will encompass, all modifications and changes apparent based on this disclosure.

We claim:

1. A foamyvirus expression system, the system comprising:
a plurality of recombinant vectors comprising an expression sequence encoding at least one component of a foamyvirus particle, wherein the expression sequence is optimized for expression in homo sapiens.

2. The foamyvirus expression system of claim 1, wherein the expression sequence is selected from a group consisting of SEQ. ID. NOS. 1-4, an optimized sequence at least about 75% identical to one of SEQ. ID. NOS. 1-4, an optimized sequence at least about 85% identical to one of SEQ. ID. NOS. 1-4, an optimized sequence at least about 95% identical to one of SEQ. ID. NOS, 1-4, and an optimized sequence at least about 99% identical to one of SEQ. ID. NOS. 1-4.

3. The foamyvirus expression system of claim 1, wherein the expression sequence is a fragment of one of SEQ. ID. NOS. 1-4, wherein the fragment has a length selected from a group consisting of at least 250 nucleotides, at least 500 nucleotides, at least 750 nucleotides, at least 1000 nucleotides, at least 1250 nucleotides, at least 1500 nucleotides, at least 1750 nucleotides, and at least 2000 nucleotides.

4. The foamyvirus expression system of claim 1, wherein the plurality of recombinant vectors comprise a first vector and a second vector, the first vector comprising an RNA packaging signal, and the second vector comprising an env expression sequence.

5. The foamyvirus expression system of claim 4, wherein the plurality of recombinant vectors comprise a third vector having a gag expression sequence.

6. The foamyvirus expression system of claim 5, wherein the third vector further comprises a pol expression sequence.

7. The foamyvirus expression system of claim 5, wherein the plurality of recombinant vectors comprise a fourth vector having a pol expression sequence.

8. The foamyvirus expression system of claim 1, wherein the plurality of recombinant vectors further comprises a non-foamyvirus expression sequence.

9. The foamyvirus expression system of claim 8, wherein the non-foamyvirus expression sequence encodes Fancg.

10. The foamyvirus expression system of claim 1, wherein the plurality of recombinant vectors are capable of producing a foamyvirus particle following transfection into an acceptable host.

11. The foamyvirus expression system of claim 10, wherein the system is capable of producing at least about 10 times as much foamyvirus particles as compared to an non-optimized foamyvirus expression system.

12. The foamyvirus expression system of claim 10, wherein the system is capable of producing at least about 100 times as much foamyvirus particles as compared to an non-optimized foamyvirus expression system.

13. The foamyvirus expression system of claim 1, wherein the at least one component of a foamyvirus particle comprises a gag protein, a pol protein, and an env protein.

14. The foamyvirus expression system of claim 13, wherein the system produces at least about 2 times as much gag protein as compared to a system having a non-optimized expression sequence.

15. The foamyvirus expression system of claim 13, wherein the system produces at least about 4 times as much gag protein as compared to a system having a non-optimized expression sequence.

16. The foamyvirus expression system of claim 13, wherein the system produces at least about 2 times as much pol protein as compared to a system having a non-optimized expression sequence.

17. The foamyvirus expression system of claim 13, wherein the system produces at least about 4 times as much pol protein as compared to a system having a non-optimized expression sequence.

18. The foamyvirus expression system of claim 13, wherein the system produces at least about 16 times as much pol protein as compared to a system having a non-optimized expression sequence.

19. The foamyvirus expression system of claim 13, wherein the system produces at least about 64 times as much pol protein as compared to a system having a non-optimized expression sequence.

20. The foamyvirus expression system of claim 13, wherein the system produces at least about 4 times as much env protein as compared to a system having a non-optimized expression sequence.

21. The foamyvirus expression system of claim 13, wherein the system produces at least about 16 times as much env protein as compared to a system having a non-optimized expression sequence.

22. The foamyvirus expression system of claim 13, wherein the system produces at least about 64 times as much env protein as compared to a system having a non-optimized expression sequence.

23. The foamyvirus expression system of claim 13, wherein the system produces at least about 128 times as much env protein as compared to a system having a non-optimized expression sequence.

24. The foamyvirus expression system of claim 13, wherein the system produces at least about 256 times as much env protein as compared to a system having a non-optimized expression sequence.

25. The foamyvirus expression system of claim 13, wherein the system produces at least about 512 times as much env protein as compared to a system having a non-optimized expression sequence.

26. A foamyvirus expression system comprising a plurality of recombinant vectors, the plurality of recombinant vectors comprising:
a first nucleotide sequence having at least a 75% nucleotide identity to SEQ. ID. NOS. 1;
a second nucleotide sequence having at least a 75% nucleotide identity to SEQ. ID. NOS. 2, and
a second nucleotide sequence having at least a 75% nucleotide identity to SEQ. ID. NOS. 3 or 4;
wherein the plurality of recombinant vectors are capable of producing a foamyvirus particle when transformed into an appropriate host cell.

27. A transformed cell comprising the foamyvirus expression system of claim 1.

28. The transformed cell of claim 27, wherein the transformed cell is capable of compensating for a genetic defect.

29. The transformed cell of claim 28, wherein the transformed cell is obtained from a patient having the genetic defect prior to transformation.

30. A kit for the expression of a foamyvirus in mammalian cells, the kit comprising;
a plurality of recombinant vectors, the plurality of recombinant vectors comprise a foamyvirus expression sequence, a first vector, a second vector, a third vector, and a fourth vector;
wherein the foamyvirus expression sequence is optimized for expression in homo sapiens;
wherein the first vector comprises a viral packaging signal, the second vector comprises a sequence encoding gag, the third vector comprises a sequence encoding pol, and the fourth vector comprises a sequence encoding env;
wherein the concentration of the first vector is greater than the second vector; the concentration of the second vector is greater than the third vector; the concentration of the third vector is greater than the fourth;
wherein the plurality of recombinant vectors produces foamyvirus particles when transfected into mammalian cells.

31. The kit of claim 31, wherein a ratio of first vector to second vector to third vector to fourth vector is about 10 to about 5 to about 1 to about 1 respectively.

32. A method of use of a foamyvirus expression system, the method comprising:
introducing a plurality of recombinant vectors having an optimized foamyvirus expression sequence into a host cell;
introducing the host cell containing the plurality of recombinant vectors into a host organism;
wherein the plurality of recombinant vectors is operable to produce foamyvirus particles after introduction into the host cell.

33. The method of claim 32, wherein the plurality of recombinant vectors is capable to compensate for a genetic defect in a host organism.

34. The method of claim 32, wherein the genetic defect results in Fanconi Anemia.
